(19) United States
(12) Patent Application Publication
(10) Pub. No.: US 2015/0166474 A1
(43) Pub. Date: Jun. 18, 2015

(54) IONICALLY BONDED SALT HAVING REACTIVE GROUP AND THERMOPLASTIC RESIN COMPOSITION CONTAINING SAME

(71) Applicant: NIPPON NYUKAZAI CO., LTD., Tokyo (JP)

(72) Inventors: Taku Murata, Kanagawa (JP); Yuta Saito, Kanagawa (JP); Taku Nakamura, Kanagawa (JP); Koichi Hotta, Kanagawa (JP)

(21) Appl. No.: 14/381,056

(22) PCT Filed: Feb. 27, 2013
(86) PCT No.: PCT/JP2013/055182

§ 371 Date: Dec. 29, 2014

(30) Foreign Application Priority Data
Feb. 29, 2012 (JP) 2012-044318
May 25, 2012 (JP) 2012-119508
Oct. 26, 2012 (JP) 2012-237048

(51) Int. Cl. C07C 309/17; C08L 23/00; C07C 229/04; C08L 67/00; C07C 305/04; C08L 33/00; C08L 25/06

(52) U.S. Cl. CPC C07C 309/17; C08L 33/00; C08L 23/00; C08L 25/06; C08L 67/00; C07C 305/04; C07C 229/04

(57) ABSTRACT

[Problem] Provided are an ionically bonded salt having a reactive group which improves the weather resistance of a thermoplastic resin, and a thermoplastic resin composition containing the ionically bonded salt.

[Solution] An ionically bonded salt which has a reactive group and is represented by the following Chemical Formula (1) or the following Chemical Formula (2):

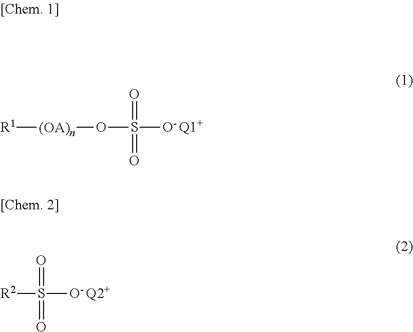

in Chemical Formulas (1) and (2) above, R¹ and R² each independently represent a substituted or unsubstituted linear, branched, or cyclic alkyl group having from 1 to 30 carbon atoms, a substituted or unsubstituted aryl group having from 6 to 30 carbon atoms, or a substituted or unsubstituted arylalkyl group having from 7 to 31 carbon atoms, A is a linear or branched alkylene group having from 2 to 4 carbon atoms, n represents an integer of 0 to 50, and Q1 and Q2 each independently represent at least one kind selected from the group consisting of an ammonium ion having an ethylenically unsaturated bond, an imidazolium ion having an ethylenically unsaturated bond, a pyridinium ion having an ethylenically unsaturated bond, a pyrrolidinium ion having an ethylenically unsaturated bond, a pyrrolinium ion having an ethylenically unsaturated bond, a piperidinium ion having an ethylenically unsaturated bond, a pyrazinium ion having an ethylenically unsaturated bond, a pyrimidinium ion having an ethylenically unsaturated bond, a triazolium ion having an ethylenically unsaturated bond, a triazinium ion having an ethylenically unsaturated bond, a quinolinium ion having an ethylenically unsaturated bond, an isoquinolinium ion having an ethylenically unsaturated bond, an indolinium ion having an ethylenically unsaturated bond, a quinoxalinium ion having an ethylenically unsaturated bond, a piperazinium ion having an ethylenically unsaturated bond, an oxazolinium ion having an ethylenically unsaturated bond, a thiazolinium ion having an ethylenically unsaturated bond, and a morpholinium ion having an ethylenically unsaturated bond.

### TECHNICAL FIELD

The present invention relates to an ionically bonded salt having a reactive group and a thermoplastic resin composition containing the same.

### BACKGROUND ART

Hitherto, it has been known that an anionic surfactant or a nonionic surfactant is used singly or in a mixture as an emulsifier for emulsion polymerization. In such a case, an alkyl sulfate salt, a polyoxyethylene alkyl ether sulfate salt, a polyoxyethylene alkylphenyl ether sulfate salt, an alkylbenzene sulfonate salt, or the like is used as the anionic surfactant, and polyoxyalkylene alkyl ether or the like is used as the nonionic surfactant. However, the emulsion obtained is poor in water resistance in a case in which the emulsion polymerization is performed using these anionic surfactants or nonionic surfactants, and thus a problem such as the peeling or adhesion failure of a coating film is caused in a case in which the emulsion is used in a paint or an adhesive.

For example, a technique has been proposed in Patent Literature 1 in which weather resistance is improved by adding a nonreactive ultraviolet absorber which does not have an unsaturated double bond in the molecule or a nonreactive hindered amine type light stabilizer to the resin for paint.

### CITATION LIST

### Patent Literature

Patent Literature 1: JP 63-43972 A

### SUMMARY OF INVENTION

### Technical Problem

However, the technique disclosed in Patent Literature 1 has a problem that the weather resistance is insufficient.

On the other hand, in order to solve the above problem, a large number of surfactants have also been proposed which contain a group having surface active ability and a reactive group in one molecule, and not only act as a monomer surfactant but also polymerize or copolymerize themselves. However, a large amount of aggregates are generated during the polymerization in a case in which these reactive group-containing surfactants are used singly. This is because the polymerization stability thereof is low, and thus there is a problem that sufficient polymerization stability cannot be obtained unless such a surfactant is concurrently used with another surfactant that does not contain a reactive group.

Accordingly, an object of the invention is to provide an ionically bonded salt having a reactive group which enables to obtain sufficient polymerization stability at the time of an emulsion polymerization and improves the water resistance or weather resistance of a thermoplastic resin, and a thermoplastic resin composition containing the ionically bonded salt.

### Means for Solving Problem

The present inventors have conducted intensive investigations in order to solve the above problem. As a result, it has been surprisingly found out that an ionically bonded salt having a specific reactive group improves the polymerization stability at the time of an emulsion polymerization and also improves the water resistance or weather resistance of a thermoplastic resin, whereby the invention has been completed.

In other words, the invention is an ionically bonded salt having a reactive group and is represented by the following Chemical Formula (1) or (2).

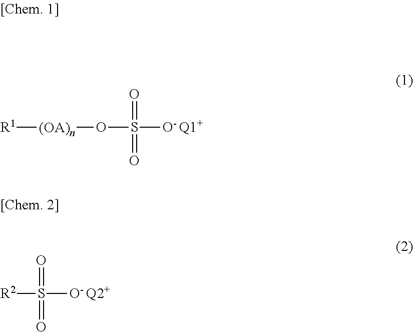

In Chemical Formulas (1) and (2) above, R¹ and R² each independently represent a substituted or unsubstituted linear, branched, or cyclic alkyl group having from 1 to 30 carbon atoms, a substituted or unsubstituted aryl group having from 6 to 30 carbon atoms, or a substituted or unsubstituted arylalkyl group having from 7 to 31 carbon atoms, A is a linear or branched alkylene group having from 2 to 4 carbon atoms, n represents an integer of 0 to 50, and Q1 and Q2 each independently represent at least one kind selected from the group consisting of an ammonium ion having an ethylenically unsaturated bond, an imidazolium ion having an ethylenically unsaturated bond, a pyridinium ion having an ethylenically unsaturated bond, a pyrrolidinium ion having an ethylenically unsaturated bond, a pyrrolinium ion having an ethylenically unsaturated bond, a piperidinium ion having an ethylenically unsaturated bond, a pyrazinium ion having an ethylenically unsaturated bond, a pyrimidinium ion having an ethylenically unsaturated bond, a triazolium ion having an ethylenically unsaturated bond, a triazinium ion having an ethylenically unsaturated bond, a quinolinium ion having an ethylenically unsaturated bond, an isoquinolinium ion having an ethylenically unsaturated bond, an indolinium ion having an ethylenically unsaturated bond, a quinoxalinium ion having an ethylenically unsaturated bond, a piperazinium ion having an ethylenically unsaturated bond, an oxazolinium ion having an ethylenically unsaturated bond, a thiazolinium ion having an ethylenically unsaturated bond, and a morpholinium ion having an ethylenically unsaturated bond.

### BRIEF DESCRIPTION OF DRAWINGS

FIG. 1 is a graph illustrating the result of foaming and antifoaming test.

### DESCRIPTION OF EMBODIMENTS

The invention is an ionically bonded salt having a reactive group and is represented by the following Chemical Formula (1) or (2).

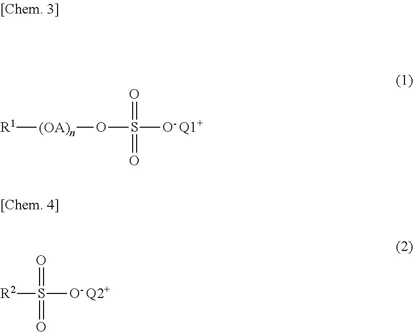

In Chemical Formulas (1) and (2) above, R¹ and R² each independently represent a substituted or unsubstituted linear, branched, or cyclic alkyl group having from 1 to 30 carbon atoms, a substituted or unsubstituted aryl group having from 6 to 30 carbon atoms, or a substituted or unsubstituted arylalkyl group having from 7 to 31 carbon atoms, A is a linear or branched alkylene group having from 2 to 4 carbon atoms, n represents an integer of 0 to 50, and Q1 and Q2 each independently represent at least one kind selected from the group consisting of an ammonium ion having an ethylenically unsaturated bond, an imidazolium ion having an ethylenically unsaturated bond, a pyridinium ion having an ethylenically unsaturated bond, a pyrrolidinium ion having an ethylenically unsaturated bond, a pyrrolinium ion having an ethylenically unsaturated bond, a piperidinium ion having an ethylenically unsaturated bond, a pyrazinium ion having an ethylenically unsaturated bond, a pyrimidinium ion having an ethylenically unsaturated bond, a triazolium ion having an ethylenically unsaturated bond, a triazinium ion having an ethylenically unsaturated bond, a quinolinium ion having an ethylenically unsaturated bond, an isoquinolinium ion having an ethylenically unsaturated bond, an indolinium ion having an ethylenically unsaturated bond, a quinoxalinium ion having an ethylenically unsaturated bond, a piperazinium ion having an ethylenically unsaturated bond, an oxazolinium ion having an ethylenically unsaturated bond, a thiazolinium ion having an ethylenically unsaturated bond, and a morpholinium ion having an ethylenically unsaturated bond. By the above configuration, it is possible to provide an ionically bonded salt having a reactive group which enables to obtain sufficient polymerization stability at the time of an emulsion polymerization and improves the water resistance or weather resistance of a thermoplastic resin, and a thermoplastic resin composition containing the ionically bonded salt.

Examples of the substituted or unsubstituted linear, branched, or cyclic alkyl group having from 1 to 30 carbon atoms which can be used as R¹ and R² in the Chemical Formulas (1) and (2) above may include a methyl group, an ethyl group, a n-propyl group, an isopropyl group, a n-butyl group, an isobutyl group, a sec-butyl group, a tert-butyl group, a n-pentyl group, an isoamyl group, a tert-pentyl group, a neopentyl group, a n-hexyl group, a 3-methylpentan-2-yl group, a 3-methylpentan-3-yl group, a 4-methylpentyl group, a 4-methylpentan-2-yl group, a 1,3-dimethylbutyl group, a 3,3-dimethylbutyl group, a 3,3-dimethylbutan-2-yl group, a n-heptyl group, a 1-methyl-hexyl group, a 3-methylhexyl group, a 4-methylhexyl group, a 5-methylhexyl group, a 1-ethylpentyl group, a 1-(n-propyl)butyl group, a 1,1-dimethylpentyl group, a 1,4-dimethylpentyl group, a 1,1-diethylpropyl group, a 1,3,3-trimethylbutyl group, a 1-ethyl-2,2-dimethylpropyl group, a n-octyl group, a 2-methylhexan-2-yl group, a 2,4-dimethylpentan-3-yl group, a 1,1-dimethylpentan-1-yl group, a 2,2-dimethylhexan-3-yl group, a 2,3-dimethylhexan-2-yl group, a 2,5-dimethylhexan-2-yl group, a 2,5-dimethylhexan-3-yl group, a 3,4-dimethylhexan-3-yl group, a 3,5-dimethylhexan-3-yl group, a 1-methylheptyl group, a 2-methylheptyl group, a 5-methylheptyl group, a 2-methylheptan-2-yl group, a 3-methylheptan-3-yl group, a 4-methylheptan-3-yl group, a 4-methylheptan-4-yl group, a 1-ethylhexyl group, a 2-ethylhexyl group, a 1-propylpentyl group, a 2-propylpentyl group, a 1,1-dimethylhexyl group, a 1,4-dimethylhexyl group, a 1,5-dimethylhexyl group, a 1-ethyl-1-methylpentyl group, a 1-ethyl-4-methylpentyl group, a 1,1,4-trimethylpentyl group, a 2,4,4-trimethylpentyl group, a 1-isopropyl-1,2-dimethylpropyl group, a 1,1,3,3-tetramethylbutyl group, a n-nonyl group, a 1-methyloctyl group, a 6-methyloctyl group, a 1-ethylheptyl group, a 1-(n-butyl)pentyl group, a 4-methyl-1-(n-propyl)pentyl group, a 1,5,5-trimethylhexyl group, a 1,1,5-trimethylhexyl group, a 2-methyloctan-3-yl group, a n-decyl group, a 1-methylnonyl group, a 1-ethyloctyl group, a 1-(n-butyl)hexyl group, a 1,1-dimethyloctyl group, a 3,7-dimethyloctyl group, a n-undecyl group, a 1-methyldecyl group, a 1-ethylnonyl group, a n-dodecyl group, a n-tridecyl group, a n-tetradecyl group, a 1-methyltridecyl group, a n-pentadecyl group, a n-hexadecyl group, a n-heptadecyl group, a n-octadecyl group, a n-nonadecyl group, a n-eicosyl group, a cyclopropyl group, a cyclobutyl group, a cyclopentyl group, a cyclohexyl group, and a cyclooc2tyl group. A methyl group, a n-propyl group, an isopropyl group, a n-butyl group, an isobutyl group, a n-hexyl group, a 2-ethylhexyl group, a n-dodecyl group, or a n-tridecyl group is preferable, and a n-dodecyl group or a n-tridecyl group is more preferable from the viewpoint of easy availability.

Examples of the substituted or unsubstituted aryl group having from 6 to 30 carbon atoms which can be used as R¹ and R² in the Chemical Formulas (1) and (2) above may include a phenyl group, a dimethylphenyl group (a 2,3-dimethylphenyl group, a 2,4-dimethylphenyl group, a 3,4-dimethylphenyl group or the like), an isopropylphenyl group (a 2-isopropylphenyl group, a 3-isopropylphenyl group, and a 4-isopropylphenyl group), a dodecylphenyl group (a 2-dodecylphenyl group, a 3-dodecylphenyl group, and a 4-dodecyl phenyl group), a cumylphenyl group, a biphenyl group, a 1-naphthyl group, a 2-naphthyl group, a 9-anthryl group, a 9-phenanthryl group, a 1-pyrenyl group, a 5-naphthacenyl group, a 1-indenyl group, a 2-azulenyl group, a 9-fluorenyl group, a terphenyl group, a quarterphenyl group, a mesityl group, a pentalenyl group, a binaphthalenyl group, a ternaphthalenyl group, a heptalenyl group, a biphenylenyl group, an indacenyl group, a fluoranthenyl group, an acenaphthylenyl group, an aceanthrylenyl group, a phenalenyl group, a fluorenyl group, an anthryl group, a bianthracenyl group, an anthraquinolyl group, a phenanthryl group, a triphenylenyl group, a pyrenyl group, a chrysenyl group, a naphthacenyl group, a pleiadenyl group, a picenyl group, a perylenyl group, a pentaphenyl group, a pentacenyl group, a tetraphenylenyl group, a hexaphenyl group, a hexacenyl group, a rubicenyl group, a coronenyl group, a trinaphthylenyl group, a heptaphenyl group, a heptacenyl group, a pyranthrenyl group, and an ovalenyl group. A substituted or unsubstituted aryl group having from 6 to 18 carbon atoms is preferable and a dimethylphenyl group (a 2,3-dimethylphenyl group, a 2,4-dimethylphenyl group, a 3,4-dimethylphenyl group, or the like), an isopropylphenyl group (a 2-isopropylphenyl group, a 3-isopropylphenyl group, and a 4-isopropylphenyl group), a dodecylphenyl group (a 2-dodecylphenyl group, a 3-dodecylphenyl group, and a 4-dodecyl phenyl group), or a cumylphenyl group is particularly preferable from the viewpoint of easy availability.

Examples of the substituted or unsubstituted arylalkyl group having from 7 to 31 carbon atoms which can be used as R¹ and R² in the Chemical Formulas (1) and (2) above may include a benzyl group, a phenylethyl group, a 3-phenylpropyl group, a 1-naphthylmethyl group, a 2-naphthylmethyl group, a 2-(1-naphthyl)ethyl group, a 2-(2-naphthyl)ethyl group, a 3-(1-naphthyl)propyl group, or a 3-(2-naphthyl)propyl group.

Examples of the linear or branched alkylene group having from 2 to 4 carbon atoms which can be used as A in the Chemical Formula (1) above may include an ethylene group, a propylene group, and a butylene group. An ethylene group or a propylene group is particularly preferable from the viewpoint of easy availability.

A hydrogen atom in the substituted or unsubstituted linear, branched, or cyclic alkyl group having from 1 to 30 carbon atoms, the substituted or unsubstituted aryl group having from 6 to 30 carbon atoms, the substituted or unsubstituted arylalkyl group having from 7 to 31 carbon atoms, and the linear or branched alkylene group having from 2 to 4 carbon atoms may be further substituted with another substituent.

Examples of such a substituent may include a hydroxyl group, a carboxyl group, a formyl group, a mercapto group, a sulfo group, a mesyl group, p-toluenesulfonyl group, an amino group, a nitro group, a cyano group, a trifluoromethyl group, a trichloromethyl group, a trimethylsilyl group, a phosphinico group, and a phosphono group in addition to a halogen such as fluorine, chlorine, bromine, and iodine, an alkyl group such as a methyl group, an ethyl group, a tert-butyl group, and a dodecyl group, an aryl group such as a phenyl group, a p-tolyl group, a xylyl group, a cumenyl group, a naphthyl group, an anthryl group, and a phenanthryl group, an alkoxy group such as a methoxy group, an ethoxy group, and a tert-butoxy group, an aryloxy group such as a phenoxy group and a p-tolyloxy group, an alkoxycarbonyl group such as a methoxycarbonyl group, a butoxycarbonyl group, 2-ethylhexyloxycarbonyl group, and a phenoxycarbonyl group, an acyloxy group such as an acetoxy group, a propionyloxy group, and a benzoyloxy group, an acyl group such as an acetyl group, a benzoyl group, an isobutyryl group, an acryloyl group, a methacryloyl group, and a methoxalyl group, an alkylsulfanyl group such as methylsulfanyl group and a tert-butylsulfanyl group, an arylsulfanyl group such as a phenylsulfanyl group and a p-tolylsulfanyl group, an alkylamino group such as a methylamino group and a cyclohexylamino group, a dialkylamino group such as a dimethylamino group, a diethylamino group, a morpholino group, and a piperidino group, an arylamino group such as a phenylamino group and a p-tolylamino group.

n in the Chemical Formula (1) above represents an integer of 0 to 50. n is preferably an integer of 1 to 50 and more preferably an integer of 5 to 50 from the viewpoint of ease of handling due to a decrease in viscosity or interfacial properties.

The cation represented by Q1 and Q2 in the Chemical Formulas (1) and (2) above is at least one kind selected from the group consisting of an ammonium ion having an ethylenically unsaturated bond, an imidazolium ion having an ethylenically unsaturated bond, a pyridinium ion having an ethylenically unsaturated bond, a pyrrolidinium ion having an ethylenically unsaturated bond, a pyrrolinium ion having an ethylenically unsaturated bond, a piperidinium ion having an ethylenically unsaturated bond, a pyrazinium ion having an ethylenically unsaturated bond, a pyrimidinium ion having an ethylenically unsaturated bond, a triazolium ion having an ethylenically unsaturated bond, a triazinium ion having an ethylenically unsaturated bond, a quinolinium ion having an ethylenically unsaturated bond, an isoquinolinium ion having an ethylenically unsaturated bond, an indolinium ion having an ethylenically unsaturated bond, a quinoxalinium ion having an ethylenically unsaturated bond, a piperazinium ion having an ethylenically unsaturated bond, an oxazolinium ion having an ethylenically unsaturated bond, a thiazolinium ion having an ethylenically unsaturated bond, and a morpholinium ion having an ethylenically unsaturated bond.

Specific examples of the cation represented by Q1 and Q2 may include a monovinylammonium ion, a divinylammonium ion, a trivinylammonium ion, a monopropenylammonium ion, a dipropenylammonium ion, a tripropenylammonium ion, a monobutenylammonium ion, a dibutenylammonium ion, a tributenylammonium ion, a monopentenylammonium ion, a dipentenylammonium ion, a tripentenylammonium ion, a monohexenylammonium ion, a dihexenylammonium ion, a monoheptenylammonium ion, a diheptenylammonium ion, a monooctenylammonium ion, a dioctenylammonium ion, a monononenylammonium ion, a monodecenylammonium ion, a monoundecenylammonium ion, a monododecenylammonium ion, a monotridecenylammonium ion, a monotetradecenylammonium ion, a monopentadecenylammonium ion, a monohexadecenylammonium ion, a monoheptadecenylammonium ion, a monooctadecenylammonium ion, a monononadecenylammonium ion, a monoicocenylammonium ion, a monohenicocenylammonium ion, a monodococenylammonium ion, a monotricocenylammonium ion, a dimethyl(vinyl)ammonium ion, a dimethyl(propenyl)ammonium ion, a dimethyl(butenyl)ammonium ion, a dimethyl(pentenyl)ammonium ion, a dimethyl(hexenyl)ammonium ion, a dimethyl(heptenyl)ammonium ion, a dimethyl(octenyl)ammonium ion, a dimethyl(nonenyl)ammonium ion, a dimethyl(decenyl)ammonium ion, a dimethyl(undecenyl)ammonium ion, a dimethyl(dodecenyl)ammonium ion, a dimethyl(tridecenyl)ammonium ion, a dimethyl(tetradecenyl)ammonium ion, a dimethyl(pentadecenyl)ammonium ion, a dimethyl(hexadecenyl)ammonium ion, a dimethyl(heptadecenyl)ammonium ion, a dimethyl(octadecenyl)ammonium ion, a dimethyl(nonadecenyl)ammonium ion, a dimethyl(icocenyl)ammonium ion, a dimethyl(henicocenyl)ammonium ion, a dimethyl(tricocenyl)ammonium ion, a dimethyl monoacrylic acid ethyl ammonium ion, a dimethyl monomethacrylic acid ethyl ammonium ion, a diethyl monoacrylic acid ethyl ammonium ion, a diethyl monomethacrylic acid ethyl ammonium ion, a 2-vinylpyridinium ion, a 4-vinylpyridinium ion, a 1,2,2,6,6-pentamethyl-4-methacrylate piperidinium ion, a 1,3,5-triacryloylhexahydro-1,3,5-triazinium ion, a 2,4,6-tris(allyloxy)-1,3,5-triazinium ion, a 2,4,6-tris(allyloxy)-1,3,5-triazinium ion, a 1-allylpiperazinium ion, a 1-(2-methylallyl)piperazinium ion, and a N-(meth)acryloylmorpholinium ion. Among them, a dimethyl monoacrylic acid ethyl ammonium ion, a dimethyl monomethacrylic acid ethyl ammonium ion, a diethyl monoacrylic acid ethyl ammonium ion, a diethyl monomethacrylic acid ethyl ammonium ion, or 1,2,2,6,6-pentamethyl-4-methacrylate piperidinium ion is more preferable.

Examples of more preferable compound of the ionically bonded salt which has a reactive group and is represented by Chemical Formula (1) or (2) above may include ionically bonded salts which has a reactive group and is represented by the following Chemical Formulas (3) to (19).

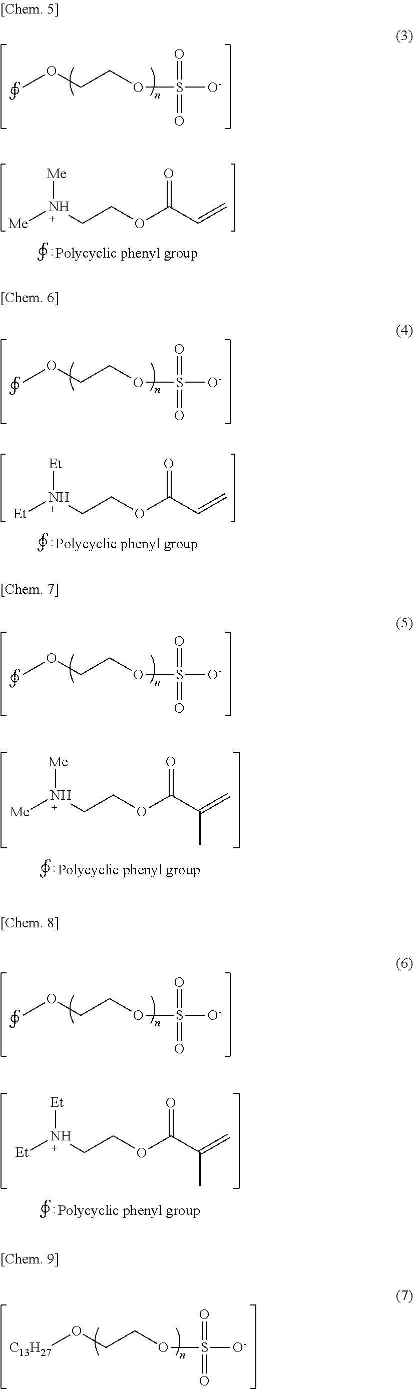

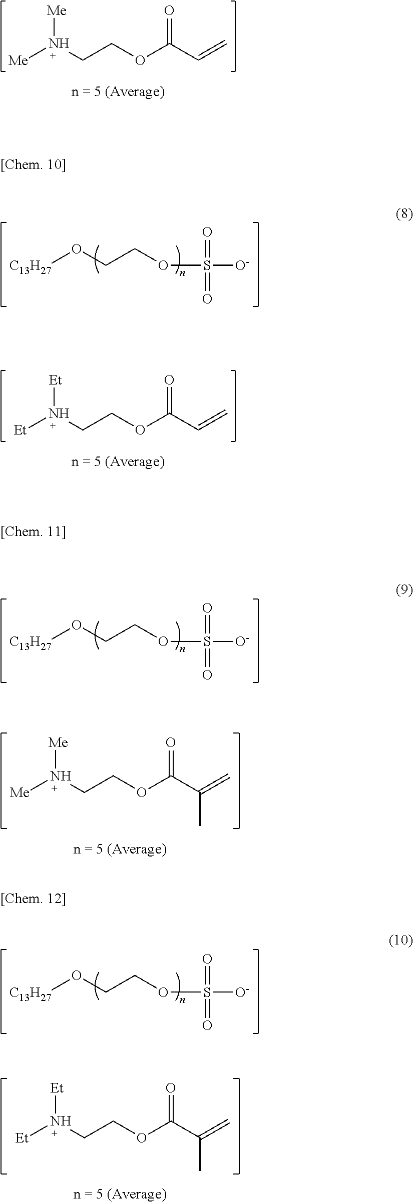

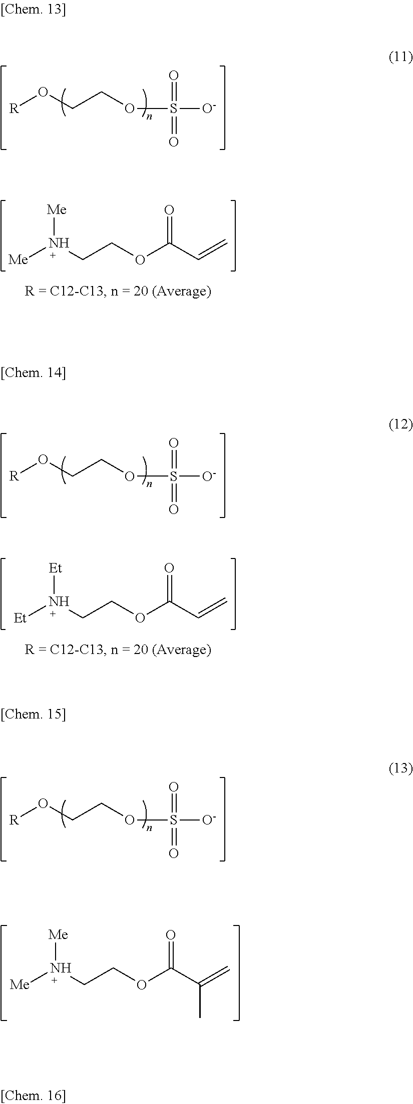

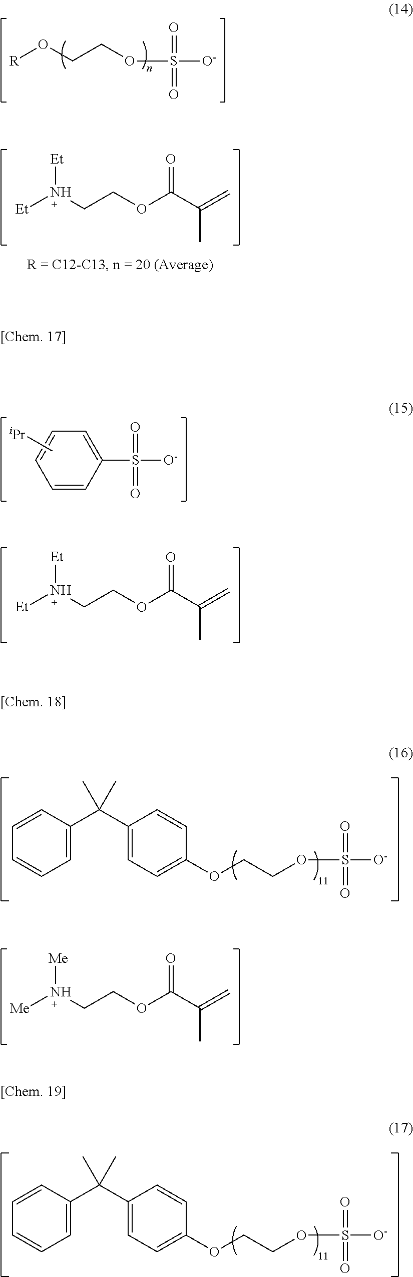

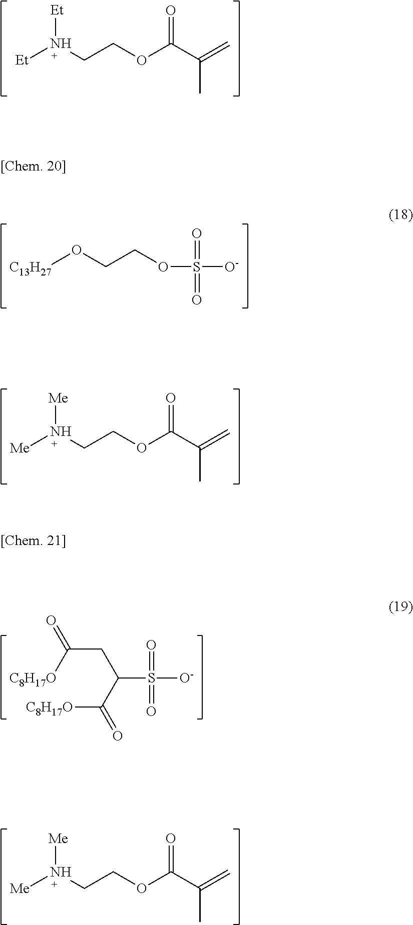

Meanwhile, the number of the reactive group contained in the ionically bonded salt of the invention is not limited, but is preferably one since an unneeded crosslinking reaction hardly occurs, for example, when the ionically bonded salt of the invention is used as an emulsifier for an emulsion polymerization and thus it becomes easier to control the molecular weight of the desired polymer.

The method of producing the ionically bonded salt having a reactive group is not particularly limited, and examples thereof may include an anion exchange method, a neutralization method, and an acid ester method. In addition, a deammoniation method in which an ammonium salt of a sulfuric acid ester or an ammonium salt of a sulfonic acid ester is allowed to react with a nitrogen-containing compound, and ammonia is removed by distillation, thereby obtaining an ionically bonded salt having a reactive group, or the like may also be suitably used.

[Treatment Agent]

The treatment agent according to the invention is a treatment agent providing a physical action or a chemical action to a plastic, a metal, a gas such as CO₂, or the like. More specifically, examples of the application of the treatment agent may include an electrolyte, a lubricant, an antistatic agent, an antifogging agent, a dispersant, an emulsifier, an acid gas absorbent, a waste water treatment agent, a synthesis catalyst, a drug delivery system (DDS), a metal processing, a polymer processing, and a weathering agent.

Hereinafter, the detailed description will be given to an antistatic agent, an antifogging agent, and a dispersing and emulsifying agent which are representative treatment agents.

(Antistatic Agent)

An antistatic agent is provided as an exemplary embodiment of a treatment agent containing the ionically bonded salt having a reactive group.

The method of using of the treatment agent containing the ionically bonded salt having a reactive group as the antistatic agent is not particularly limited, and examples thereof may include a method in which the antistatic agent is mixed with the starting material for producing the molded article to be imparted with antistatic properties or is coated on the molded article to be imparted with antistatic properties. Here, the molded article of which the antistatic properties can be adjusted using the treatment agent containing the ionically bonded salt having a reactive group is not particularly limited, and examples thereof may include plastic, glass, fiber, sheet or tape, preferably plastic or glass, and particularly preferably plastic.

Examples of the plastic may include a polyacrylate-based resin, a polystyrene-based resin, a polyester-based resin, a polypropylene-based resin, a polycarbonate-based resin, a polyamide-based resin, and a polyacrylate-styrene-based resin of a copolymer of these, preferably a polyacrylate-based resin, a polyester-based resin, and a polyacrylate-styrene-based resin, and more preferably a polyester-based resin.

The content of the ionically bonded salt having a reactive group in the treatment agent is preferably from 0.01 to 20 parts by mass and more preferably from 1 to 10 parts by mass when the entire treatment agent is set to 100 parts by mass from the viewpoint of economy and/or aesthetic sense in a case in which the treatment agent is used as an antistatic agent.

Examples of a component other than the ionically bonded salt having a reactive group contained in the treatment agent may include an inorganic-based or organic-based filler, various kinds of surfactants, a reactive compound such as a polyvalent isocyanate compound, an epoxy compound, and an organometallic compound, an antioxidant, a silicone oil, a processing aid, a ultraviolet absorber, a fluorescent brightening agent, an antislip agent, an anti-blocking agent, an antifogging agent, a light stabilizer, a lubricant, a softener, a colored dye, and another stabilizer in a case in which the treatment agent is used as an antistatic agent.

The single substance of the ionically bonded salt having a reactive group in the treatment agent is preferably contained from 0.5 to 20 parts by mass and particularly preferably from 1 to 10 parts by mass with respect to 100 parts by mass of the resin contained in the starting material of the molded article in a case in which the treatment agent is used by being mixed with the starting material for producing the molded article to be imparted with antistatic properties.

In addition, a known coating method can be appropriately used as the method of coating the treatment agent in a case in which the treatment agent is used by being coated on the molded article to be imparted with antistatic properties, and for example, a method such as spin coating, roll coating, gravure coating, reverse coating, spray coating, air knife coating, curtain coating, roll brush, or impregnation can be used.

It is considered that the antistatic effect obtained by the treatment agent of the invention is attributed to a high ionic conductivity or electrical conductivity of the ionically bonded salt having a reactive group contained in the antistatic agent as a factor.

(Antifogging Agent)

An antifogging agent is provided as an exemplary embodiment of a treatment agent containing the ionically bonded salt having a reactive group.

The method of using of the treatment agent containing the ionically bonded salt having a reactive group as the antifogging agent is not particularly limited, and examples thereof may include a method in which the antifogging agent is mixed with the starting material for producing the molded article to be imparted with antifog property or is coated on the molded article to be imparted with antifog property. Here, the molded article of which the antifog property can be adjusted using the treatment agent containing the ionically bonded salt having a reactive group is not particularly limited, and examples thereof may include plastic, glass, fiber, sheet or tape, and preferably plastic or glass.

The content of the ionically bonded salt having a reactive group in the treatment agent is preferably from 0.5 to 20 parts by mass and particularly preferably from 1 to 10 parts by mass when the entire treatment agent is set to 100 parts by mass from the viewpoint of economy and/or aesthetic sense in a case in which the treatment agent is used as an antifogging agent.

Examples of a component other than the ionically bonded salt having a reactive group contained in the treatment agent may include an inorganic-based or organic-based filler, various kinds of surfactants, a reactive compound such as a polyvalent isocyanate compound, an epoxy compound, and an organometallic compound, an antioxidant, a silicone oil, a processing aid, a ultraviolet absorber, a fluorescent brightening agent, an antislip agent, an anti-blocking agent, an antistatic agent, a light stabilizer, a lubricant, a softener, a colored dye, and another stabilizer in a case in which the treatment agent is used as an antifogging agent.

The single substance of the ionically bonded salt having a reactive group in the treatment agent is preferably contained from 0.5 to 20 parts by mass and particularly preferably from 1 to 10 parts by mass with respect to 100 parts by mass of the resin contained in the starting material of the molded article in a case in which the treatment agent is used by being mixed with the starting material for producing the molded article to be imparted with antifog property.

In addition, a known coating method can be appropriately used as the method of coating the treatment agent in a case in which the treatment agent is used by being coated on the molded article to be imparted with antifog property, and for example, a method such as spin coating, roll coating, gravure coating, reverse coating, spray coating, air knife coating, curtain coating, roll brush, or impregnation can be used.

It is considered that the antifog effect obtained by the treatment agent of the invention is attributed to the fact that the ionically bonded salt having a reactive group contained in the treatment agent decreases the contact angle of water drops attached to the surface of the molded article and thus the formation of sparse water drops which are the cause of the cloudiness is suppressed as a factor.

(Dispersant and Emulsifier)

A dispersant and emulsifier are provided as an exemplary embodiment of a treatment agent containing the ionically bonded salt having a reactive group.

The method of using of the treatment agent containing the ionically bonded salt having a reactive group as a dispersant is not particularly limited, and examples thereof may include a method in which the dispersant is used as a cleaning agent, a dyeing auxiliary, a softener, an anti-scattering agent, an asphalt emulsion, a cement slurry viscosity reducing agent, a concrete air-entraining agent, a wetting agent, a paraffin emulsifier, a felt cleaning agent, a water dispersible powder, a preservative, an antistatic agent, a hydrophilicity imparting agent, a fungicide, or the like.

The method of using the treatment agent containing the ionically bonded salt having a reactive group as an emulsifier is not particularly limited, and examples thereof may include a method in which the treatment agent is added dropwise to the monomer for emulsion polymerization, or mixed with the monomer little by little or collectively.

The preferred monomer used in the emulsion polymerization is not particularly limited, and examples thereof may include an acrylate-based monomer, a styrene-based monomer, an allyl monomer, and vinyl monomer, and the monomers may be used in a mixture. An acrylate-based monomer and a mixture of an acrylate monomer and a styrene-based monomer are preferable.

The content of the ionically bonded salt having a reactive group in the treatment agent is preferably from 0.1 to 20 parts by mass and more preferably from 0.5 to 5 parts by mass when the entire treatment agent is set to 100 parts by mass.

In a case in which a molded article is produced using the resin obtained by a suspension polymerization or an emulsion polymerization using the treatment agent containing the ionically bonded salt having a reactive group as a dispersant and emulsifier, the molded article thus produced may have a weather resistance and water resistance. It is considered that the effect of improving weather resistance and water resistance of the molded article is attributed to the fact that the ionically bonded salt having a reactive group has an organic cation as a factor. Specifically, the ionically bonded salt having a reactive group according to the invention has an organic cation exhibiting relatively weak repulsive force between the organic cations in comparison with a normal dispersant and emulsifier which have a cation exhibiting strong repulsive force between ions such as a sodium ion. Hence, the particle size of the resin obtained by a suspension polymerization, an emulsion polymerization, or the like may be smaller. A coating film exerts high adhesive property when a molded article is produced by coating such a resin having a small particle size on, for example, plastic, and thus it is considered that the molded article thus produced may have a weather resistance and water resistance.

The ionically bonded salt contained in the treatment agent is preferably from 10 to 100 parts by mass when the entire treatment agent is set to 100 parts by mass.

It is considered that the effect of lubricant obtained by the treatment agent of the invention is attributed to the heat resistance and nonvolatility of the ionically bonded salt contained in the lubricant as a treatment agent of the invention as a factor.

The ionically bonded salt having a reactive group contained in the treatment agent is preferably from 10 to 100 parts by mass when the entire treatment agent is set to 100 parts by mass.

Moreover, the ionically bonded salt having a reactive group of the invention can be suitably used as a weathering agent of a thermoplastic resin.

In other words, the invention provides a thermoplastic resin composition containing the ionically bonded salt having a reactive group and a thermoplastic resin.

[(a) Thermoplastic Resin]

The (a) thermoplastic resin used in the invention is not particularly limited, but examples thereof may include a (meth)acrylic resin, a styrene resin, an olefin resin (including a cyclic olefin resin), a polyester resin, a polycarbonate resin, a polyamide resin, a polyphenylene ether resin, a polyphenylene sulfide resin, a halogen-containing resin (polyvinyl chloride, polyvinylidene chloride, fluororesin, or the like), a polysulfone resin (polyethersulfone, polysulfone, or the like), a cellulose derivative (a cellulose ester, a cellulose carbamate, a cellulose ether, or the like), a silicone resin (polydimethylsiloxane, polymethylphenylsiloxane, or the like), a polyvinyl ester resin such as polyvinyl acetate, a polyvinyl alcohol resin and a derivative resin thereof, and a rubber or an elastomer (a diene rubber such as polybutadiene and polyisoprene, a styrene-butadiene copolymer, an acrylonitrile-butadiene copolymer, an acrylonitrile-butadiene-styrene copolymer (ABS resin), an acrylic rubber, a urethane rubber, a silicone rubber, or the like). The thermoplastic resins above can be used singly or in combination of two or more kinds thereof.

Specific examples of the (meth)acrylic resin may include (meth)acrylic acid, a (meth)acrylic acid ester [a (meth)acrylic acid alkyl ester having an alkyl group having from 1 to 10 carbon atoms such as methyl (meth)acrylate, ethyl(meth)acrylate, propyl(meth)acrylate, butyl (meth)acrylate, and 2-ethylhexyl(meth)acrylate; a (meth)acrylic acid hydroxyalkyl such as hydroxyethyl(meth)acrylate; a (meth)acrylic acid glycidyl ester; or the like], a homopolymer or copolymer of a (meth)acrylic monomer such as (meth)acrylonitrile; and a copolymer of a (meth)acrylic monomer and another monomer.

Specific examples of the homopolymer or copolymer of a (meth)acrylic monomer may include a poly(meth)acrylic acid ester, an acrylic acid ester-methacrylic acid ester copolymer, and polyacrylonitrile. Specific examples of the copolymer of a (meth)acrylic monomer and another monomer may include a (meth)acrylic acid-styrene copolymer, a (meth)acrylic acid ester-styrene copolymer, a (meth)acrylic acid ester-(meth)acrylic acid-styrene copolymer, an acrylonitrile-butadiene-styrene copolymer, an acrylonitrile-styrene copolymer, an acrylonitrile-styrene-(meth)acrylic acid ester copolymer, an acrylonitrile-acrylic acid ester-styrene copolymer (AAS resin), and a methyl methacrylate-butadiene-styrene copolymer (MBS resin).

Specific examples of the styrene resin may include polystyrene, poly-α-methylstyrene, an α-methylstyrene-acrylonitrile copolymer, a styrene-N-phenylmaleimide copolymer, a styrene-N-phenylmaleimide-acrylonitrile copolymer, and a rubber-reinforced polystyrene resin (HIPS resin).

As the olefin resin, a copolymer of an olefin monomer, a copolymer of an olefin monomer and another copolymerizable monomer in addition to a homopolymer of an olefin monomer may be included. Specific examples of the olefin monomer may include a chain olefin [an α-olefin having from 2 to 20 carbon atoms such as ethylene, propylene, 1-butene, isobutene, 1-pentene, 4-methyl-1-pentene, 1-hexene, 1-octene, 1-decene, or the like] and a cyclic olefin [for example, a cycloalkene having from 4 to 10 carbon atoms such as cyclopentene; a cycloalkadiene having from 4 to 10 carbon atoms such as cyclopentadiene; a bicycloalkene having from 7 to 20 carbon atoms or a bicycloalkadiene having from 7 to 20 carbon atoms such as norbornene and norbornadiene; and a tricycloalkene having from 10 to 25 carbon atoms or a tricycloalkadiene such as dihydrodicyclopentadiene and dicyclopentadiene]. These olefin monomers can be used singly or in combination of two or more kinds thereof. Among the olefin monomers above, a chain olefin such as an α-olefin having from 2 to 4 carbon atoms including ethylene, propylene, and 1-butene is preferable.

Specific examples of another copolymerizable monomers copolymerizable with the olefin monomer may include a fatty acid vinyl ester such as vinyl acetate and vinyl propionate; a (meth)acrylic monomer such as (meth)acrylic acid, an alkyl(meth)acrylate, and glycidyl(meth)acrylate; an unsaturated dicarboxylic acid or an anhydride thereof such as maleic acid, fumaric acid, and maleic anhydride; a vinyl ester of a carboxylic acid (for example, vinyl acetate, vinyl propionate, or the like); a cyclic olefin such as norbornene and cyclopentadiene; and a diene such as butadiene and isoprene. These copolymerizable monomers may be used singly or in combination of two or more kinds thereof.

More specific examples of the olefin resin may include polyethylene (low density polyethylene, medium density polyethylene, high density polyethylene, linear low density polyethylene, or the like), polypropylene (homo polypropylene, block polypropylene, random polypropylene, or the like), and a (co)polymer of a chain olefin (particularly an α-olefin having from 2 to 4 carbon atoms) such as a terpolymer including an ethylene-propylene copolymer and an ethylene-propylene-butene-1. In addition, specific examples of the copolymer of an olefin monomer and another copolymerizable monomer may include a copolymer (for example, an ethylene-vinyl acetate copolymer, an ethylene-vinyl propionate copolymer, or the like) of a chain olefin (particularly an α-olefin having from 2 to 4 carbon atoms such as ethylene and propylene) and a fatty acid vinyl ester monomer; a copolymer [a copolymer (for example, an ethylene-(meth)acrylic acid copolymer, a propylene-(meth)acrylic acid copolymer, ionomer, or the like) of a chain olefin (particularly an α-olefin having from 2 to 4 carbon atoms) and (meth)acrylic acid; a copolymer (for example, an ethylene-alkyl(meth)acrylate copolymer, or the like) of a chain olefin (particularly an α-olefin having from 2 to 4 carbon atoms) and an alkyl(meth)acrylate; or the like] of a chain olefin and a (meth)acrylic monomer; a copolymer (for example, an ethylene-butadiene copolymer, or the like) of a chain olefin (particularly an α-olefin having from 2 to 4 carbon atoms) and a diene; a modified polyolefin such as an epoxy-modified polyolefin (for example, an ethylene-glycidyl(meth)acrylate copolymer), a carboxy-modified polyolefin (for example, an ethylene-maleic anhydride copolymer), and an epoxy and carboxy-modified polyolefin (for example, an ethylene-maleic anhydride-glycidyl(meth)acrylate copolymer); and an olefin elastomer (an ethylene-propylene rubber, or the like).

Specific examples of the polyester resin may include a polymer or a copolymer obtained through the polycondensation of at least one kind selected from the group consisting of (i) a dicarboxylic acid or a derivative thereof and a diol or a derivative thereof, (ii) a hydroxy carboxylic acid or a derivative thereof, and (iii) a lactone.

Examples of the dicarboxylic acid or the derivative thereof may include an aromatic dicarboxylic acid such as terephthalic acid, isophthalic acid, phthalic acid, 2,6-naphthalenedicarboxylic acid, 1,5-naphthalen dicarboxylic acid, bis(p-carboxyphenyl)methane, anthracenedicarboxylic acid, 4,4′-diphenyl ether dicarboxylic acid, 5-tetrabutylphosphoniumisophthalic acid, and 5-sodiosulfoisophthalic acid, an aliphatic dicarboxylic acid such as oxalic acid, succinic acid, adipic acid, sebacic acid, azelaic acid, dodecanedioic acid, malonic acid, glutaric acid, and dimer acid, 1,3-cyclohexanedicarboxylic acid, and 1,4-cyclohexanedicarboxylic acid. Examples of the diol or the derivative thereof may include an aliphatic glycol having from 2 to 20 carbon atoms, that is, ethylene glycol, propylene glycol, 1,4-butanediol, neopentyl glycol, 1,5-pentanediol, 1,6-hexanediol, decamethylene glycol, cyclohexane dimethanol, cyclohexane diol, dimer diol, or the like, or a long chain glycol having a molecular weight of from 200 to 100,000, that is, polyethylene glycol, poly-1,3-propylene glycol, polytetramethylene glycol, or the like, an aromatic dioxy compound, that is, 4,4′-dihydroxybiphenyl, hydroquinone, t-butylhydroquinone, bisphenol A, bisphenol S, bisphenol F, or the like, and a derivative thereof. Examples of the hydroxy carboxylic acid may include glycolic acid, lactic acid, hydroxypropionic acid, hydroxybutyric acid, hydroxyvaleric acid, hydroxycaproic acid, hydroxybenzoic acid, p-hydroxybenzoic acid, 6-hydroxy-2-naphthoic acid, and a derivative thereof. Examples of the lactone may include caprolactone, valerolactone, propiolactone, undecalactone, 1,5-oxepane-2-one. In addition, a polyester elastomer is also included in the polyester resin.

Specific examples of a polycarbonate resin may include a thermoplastic resin obtained through the reaction of a di- or higher valent phenolic compound with a carbonic acid diester compound such as phosgene or diphenyl carbonate.

Examples of the di- or higher valent phenolic compound may include a dihydroxydiarylalkane such as 2,2-bis(4-hydroxyphenyl)propane (commonly called as bisphenol A), bis(4-hydroxyphenyl)methane, bis(4-hydroxyphenyl)phenyl methane, bis(4-hydroxyphenyl)naphthyl methane, bis(4-hydroxyphenyl)-(4-isopropylphenyl)methane, bis(3,5-dichloro-4-hydroxyphenyl)methane, bis(3,5-dimethyl-4-hydroxyphenyl)methane, 1,1-bis(4-hydroxyphenyl)ethane, 1-naphthyl-1,1-bis(4-hydroxyphenyl)ethane, 1-phenyl-1,1-bis(4-hydroxyphenyl)ethane, 1,2-bis(4-hydroxyphenyl)ethane, 2-methyl-1,1-bis(4-hydroxyphenyl)propane, 2,2-bis(3,5-dimethyl-4-hydroxyphenyl)propane, 1-ethyl-1,1-bis(4-hydroxyphenyl)propane, 2,2-bis(3,5-dichloro-4-hydroxyphenyl)propane, 2,2-bis(3,5-dibromo-4-hydroxyphenyl)propane, 2,2-bis(3-chloro-4-hydroxyphenyl)propane, 2,2-bis(3-methyl-4-hydroxyphenyl)propane, 2,2-bis(3-fluoro-4-hydroxyphenyl)propane, 1,1-bis(4-hydroxyphenyl)butane, 2,2-bis(4-hydroxyphenyl)butane, 1,4-bis(4-hydroxyphenyl)butane, 2,2-bis(4-hydroxyphenyl)pentane, 4-methyl-2,2-bis(4-hydroxyphenyl)pentane, 2,2-bis(4-hydroxyphenyl)hexane, 4,4-bis(4-hydroxyphenyl)heptane, 2,2-bis(4-hydroxyphenyl)nonane, 1,10-bis(4-hydroxyphenyl)decane, 1,1-bis(4-hydroxyphenyl)-3,3,5-trimethyl cyclohexane, and 2,2-bis(4-hydroxyphenyl)-1,1,1,3,3,3-hexafluoropropane, a dihydroxydiaryl cycloalkane such as 1,1-bis(4-hydroxyphenyl)cyclohexane, 1,1-bis(3,5-dichloro-4-hydroxyphenyl)cyclohexane, and 1,1-bis(4-hydroxyphenyl)cyclodecane, a dihydroxydiaryl sulfone such as bis(4-hydroxyphenyl)sulfone, bis(3,5-dimethyl-4-hydroxyphenyl)sulfone, and bis(3-chloro-4-hydroxyphenyl)sulfone, a dihydroxyaryl ether such as bis(4-hydroxyphenyl)ether and bis(3,5-dimethyl-4-hydroxyphenyl)ether, a dihydroxydiaryl ketone such as 4,4′-dihydroxybenzophenone and 3,3′,5,5′-tetramethyl-4,4′-dihydroxybenzophenone, a dihydroxydiaryl sulfide such as bis(4-hydroxyphenyl)sulfide, bis(3-methyl-4-hydroxyphenyl)sulfide, and bis(3,5-dimethyl-4-hydroxyphenyl)sulfide, a dihydroxydiaryl sulfoxide such as bis(4-hydroxyphenyl)sulfoxide, a dihydroxydiphenyl such as 4,4′-dihydroxydiphenyl, and a dihydroxyaryl fluorene such as 9,9-bis(4-hydroxyphenyl)fluorene. In addition, a dihydroxybenzene such as hydroquinone, resorcinol, and methylhydroquinone, and a dihydroxynaphthalene such as 1,5-dihydroxynaphthalene and 2,6-dihydroxynaphthalene other than the divalent phenol compounds described above can be used as the divalent phenol compound.

These di- or higher valent phenol compounds may be used singly or in combination of two or more kinds thereof. In addition, a linear aliphatic dicarboxylic acid such as adipic acid, pimelic acid, suberic acid, azelaic acid, sebacic acid, and decane dicarboxylic acid may be used as a copolymerization component.

Specific examples of a polyamide resin may include an aliphatic polyamide such as polyamide 46, polyamide 5, polyamide 6, polyamide 66, polyamide 610, polyamide 612, polyamide 11, polyamide 12, polyamide 6/66, and polyamide 6/11; an alicyclic polyamide such as poly-1,4-norbornene terephthalamide, poly-1,4-cyclohexane terephthalamide, and poly-1,4-cyclohexane-1,4-cyclohexaneamide; an aromatic polyamide such as polyamide 6T, polyamide 9T, and polyamide MXD; and a copolyamide formed by at least two different kinds of polyamide-forming components among these polyamides. Meanwhile, a polyamide elastomer is also included in the polyamide resin.

Specific examples of a polyphenylene ether resin may include a homopolymer such as poly(2,5-dimethyl-1,4-phenylene ether), poly(2,6-dimethyl-1,4-phenylene ether), poly(2-methyl-6-ethyl-1,4-phenylene ether), poly(2,6-di-n-propyl-1,4-phenylene ether), and poly(2-methyl-6-chloroethyl-1,4-phenylene ether), a modified polyphenylene ether copolymer constituted by having these homopolymers as a base, and a modified graft copolymer in which a styrene polymer is grafted to a polyphenylene ether homopolymer or a copolymer thereof.

Specific examples of a polyphenylene sulfide resin may include a polyphenylene sulfide, a polyphenylene sulfide ketone, a polybiphenylene sulfide, and a polyphenylene sulfide sulfone.

Meanwhile, the form of the copolymer may be any of a block copolymer, a random copolymer, a graft copolymer, and an alternating copolymer in a case in which the thermoplastic resin described above is a copolymer.

A synthetic compound or a commercially available product may be used as the thermoplastic resin described above. The polymerization method for the synthesis of these thermoplastic resins is not particularly limited, and a known method can be used. Examples thereof may include a high-pressure radical polymerization method, a medium/low-pressure polymerization method, a solution polymerization method, a slurry polymerization method, a bulk polymerization method, an emulsion polymerization method, and a vapor phase polymerization method. In addition, the catalyst used in the polymerization is also not particularly limited, and examples thereof may include a peroxide catalyst, the Ziegler-Natta catalyst, and a metallocene catalyst.

The thermoplastic resin described above is preferably at least one kind selected from the group consisting of a (meth)acrylic resin, a styrene resin, an olefin resin, and a polyester resin.

The content of the ionically bonded salt having a reactive group in the thermoplastic resin composition of the invention is preferably from 0.1 to 20 parts by mass and more preferably from 0.5 to 5 parts by mass with respect to 100 parts by mass of the thermoplastic resin. A thermoplastic resin composition excellent in weather resistance can be obtained when the content is in this range. Meanwhile, the ionically bonded salts having a reactive group described above can be used singly or in combination of two or more kinds thereof.

The thermoplastic resin composition of the invention may be appropriately blended with another additive component within a range in which the object of the invention is not impaired. Examples of the additive component may include an antioxidant, a filler, a lubricant, a dye, an organic pigment, an inorganic pigment, a plasticizer, a processing aid, a ultraviolet absorber, a light stabilizer, a blowing agent, a wax, a nucleating agent, a mold release agent, a hydrolysis inhibitor, an anti-blocking agent, an antistatic agent, a radical scavenger, an antifogging agent, a scratch preventing agent, an ion trapping agent, a flame retardant, an auxiliary flame retardant, and a surfactant.

The form of the thermoplastic resin composition of the invention is not particularly limited, and the thermoplastic resin composition may be in any form of, for example, a solid, an emulsion, a semi-clear solution, and a clear solution, and is preferably an emulsion from the viewpoint of environmental impact reduction.

[Method of Producing Thermoplastic Resin Composition]

The method of producing the thermoplastic resin composition of the invention is not particularly limited, and examples thereof may include a method in which the ionically bonded salt having a reactive group, the thermoplastic resin, and another additive if necessary are melt-kneaded. The method of melt-kneading is not particularly limited, and it is possible to adopt a method which uses a device such as a single screw extruder, a twin screw extruder, a heat roll, the Banbury mixer, the Henschel mixer, a tumbler mixer, or various kinds of kneaders.

In addition, a method in which the composition of the invention is obtained by performing an emulsion polymerization of a monomer which is a starting material of the thermoplastic resin in an aqueous solvent using the ionically bonded salt having a reactive group of the invention as an emulsifier in the presence of a polymerization initiator, or a method in which the composition of the invention is obtained by performing a solution polymerization or a ultraviolet polymerization of the ionically bonded salt having a reactive group of the invention and a monomer which is a starting material of the thermoplastic resin in an organic solvent in the presence of a polymerization initiator can also be suitably used in terms of excellent productivity thereof. At this time, another surfactant such as an anionic surfactant or a nonionic surfactant can be used together with the ionically bonded salt having a reactive group described above as an emulsifier.

[Application of Thermoplastic Resin Composition]

The application of the thermoplastic resin composition of the invention may include, for example, a paint, an adhesive, a pressure sensitive adhesive, a textile auxiliary, a paper making application (a surface coating agent, or the like), a civil engineering application (such as an admixture for concrete, or the like), and the like.

### EXAMPLES

Hereinafter, the invention will be described in more detail by reference to Examples, but the invention is not intended to be limited in any way by the following Examples. Meanwhile, the indication “part” or “%” used in some cases below represent “part by mass” or “% by mass” unless otherwise stated.

### Synthesis Example 1

### Synthesis of [2A-MA] [707-5]

To 185.3 parts by mass of N,N-diethylaminoethyl methacrylate (abbreviation: 2A-MA), 920.1 parts by mass of ammonium salt of polyoxyethylene polycyclic phenyl ether sulfuric acid ester (abbreviation: 707-SF) obtained by removing water from a 30% aqueous solution of ammonium salt of polyoxyethylene polycyclic phenyl ether sulfuric acid ester (trade name: NEWCOL 707-SF, abbreviation: N-707-SF manufactured by Nippon Nyukazai Co., Ltd.) by distillation under reduced pressure was added, and then the resultant was allowed to react for 1 hour. After the reaction, 1088.4 parts by mass of the desired ionically bonded salt (abbreviation: [2A-MA] [707-S], the compound represented by the Chemical Formula (6) above) was obtained.

### Synthesis Example 2

### Synthesis of [2Mabs-MA] [707-S]

In the same manner as in the Synthesis Example 1 except that 157.2 parts by mass of N,N-dimethylaminoethyl methacrylate (abbreviation: 2Mabs-MA) was used instead of 2A-MA, 1060.3 parts by mass of the desired ionically bonded salt (abbreviation: [2Mabs-MA] [707-5], the compound represented by the Chemical Formula (5) above) was obtained.

### Synthesis Example 3

### Synthesis of [2Mabs-A] [707-S]

In the same manner as in the Synthesis Example 1 except that 143.2 parts by mass of N,N-dimethylaminoethyl acrylate (abbreviation: 2Mabs-A) was used instead of 2A-MA, 1046.3 parts by mass of the desired ionically bonded salt (abbreviation: [2Mabs-A] [707-5], the compound represented by the Chemical Formula (3) above) was obtained.

### Synthesis Example 4

### Synthesis of [2A-MA] [1305-5]

In the same manner as in the Synthesis Example 1 except that 517.4 parts by mass of ammonium salt of polyoxyethylene tridecyl ether sulfuric acid ester (abbreviation: 1305-SF), synthesized by a known method of a related art, was used instead of 707-SF, 685.7 parts by mass of the desired ionically bonded salt (abbreviation: [2A-MA] [1305-5], the compound represented by the Chemical Formula (10) above) was obtained.

### Synthesis Example 5

### Synthesis of [2Mabs-MA] [1305-S]

In the same manner as in the Synthesis Example 2 except that 517.4 parts by mass of 1305-SF was used instead of 707-SF, 657.6 parts by mass of the desired ionically bonded salt (abbreviation: [2Mabs-MA] [1305-S], the compound represented by the Chemical Formula (9) above) was obtained.

### Synthesis Example 6

### Synthesis of [2A-MA] [2320-S]

In the same manner as in the Synthesis Example 1 except that 1170.0 parts by mass of ammonium salt of plyoxyethylene alkyl ether sulfuric acid ester (abbreviation: 2320-SF), synthesized by a known method of the related art, was used instead of 707-SF, 1338.3 parts by mass of the desired ionically bonded salt abbreviation: [2A-MA] [2320-S], the compound represented by the Chemical Formula (14) above) was obtained.

### Synthesis Example 7

### Synthesis of [2Mabs-MA] [2320-S]

In the same manner as in the Synthesis Example 2 except that 1170.0 parts by mass of 2320-SF was used instead of 707-SF, 1310.2 parts by mass of the desired ionically bonded salt (abbreviation: [2Mabs-MA] [2320-S], the compound represented by the Chemical Formula (13) above) was obtained.

### Synthesis Example 8

### Synthesis of [2A-MA] [CMP11-S]

In the same manner as in the Synthesis Example 1 except that 801.0 parts by mass of ammonium salt of polyoxyethylene cumylphenol sulfuric acid ester (abbreviation: CMP11-SF) was used instead of 707-SF, 969.3 parts by mass of the desired ionically bonded salt (abbreviation: [2A-MA] [CMP11-S], the compound represented by the Chemical Formula (17) above) was obtained.

### Synthesis Example 9

### Synthesis of [2Mabs-MA] [CMP11-S]

In the same manner as in the Synthesis Example 2 except that 801.0 parts by mass of CMP11-SF was used instead of 707-SF, 941.2 parts by mass of the desired ionically bonded salt (abbreviation: [2Mabs-MA] [CMP11-S], the compound represented by the Chemical Formula (16) above) was obtained.

### Synthesis Example 10

### Synthesis of [2Mabs-AA] [1305-S]

In the same manner as in the Synthesis Example 4 except that 143.2 parts by mass of N,N-diethylaminoethyl acrylate (abbreviation: 2Mabs-AA) was used instead of 2A-MA, 643.6 parts by mass of the desired ionically bonded salt (abbreviation: [2Mabs-AA] [1305-S], the compound represented by the Chemical Formula (18) above) was obtained.

### Synthesis Example 11

### Synthesis of [2Mabs-AA] [DOSS]

Into an autoclave, 200.0 parts by mass of dioctyl fumarate, 116.4 parts by mass of a 50% aqueous solution of ammonium bisulfite, 37.0 parts by mass of methanol, and 15.6 parts by mass of water were introduced, the temperature was raised to 120° C., and then the resultant was allowed to react for 24 hours. At this time, the maximum pressure was 0.25 MPa.

Water and methanol were removed from the ammonium dioctylsulfosuccinate solution obtained in the above example by distillation under reduced pressure, thereby obtaining ammonium dioctylsulfosuccinate (abbreviation: DOSS-NH₄).

In the same manner as in the Synthesis Example 10 except that 439.6 parts by mass of DOSS-NH₄ was used instead of 1305-SF, 565.8 parts by mass of the desired ionically bonded salt (abbreviation: [2Mabs-AA] [DOSS], the compound represented by the Chemical Formula (19) above) was obtained.

### Example 1

Into a flask equipped with a condenser tube, a nitrogen inlet tube, a thermometer, a semicircular Teflon stirring blade, 100.0 parts by mass of water was introduced and heated to 80° C. Separately, 100.0 parts by mass of n-butyl acrylate, 70.0 parts by mass of methyl methacrylate, 30.0 parts by mass of styrene, 4.0 parts by mass of acrylic acid, 4.08 parts by mass of [2A-MA] [707-S] synthesized in the Synthesis Example 1 above as an emulsifier, 95.8 parts by mass of water, and 10.2 parts by mass of a 10% aqueous solution of ammonium peroxodisulfate were introduced into an Erlenmeyer flask and mixed to obtain a mixture. This mixture was introduced into the above flask equipped with a condenser tube and the like by 15.7 parts by mass, and the temperature thereof was raised to 80° C. and the initial polymerization was performed for 30 minutes. Thereafter, the mixture in the Erlenmeyer flask was added into the resultant in the flask equipped with a condenser tube and the like dropwise at 80° C. over 3 hours. Further, aging of the resultant was performed at 80° C. for 1 hour, and then neutralization of the resultant was performed using 0.8 part by mass of 28% ammonia water to obtain an emulsion.

### Example 2

An emulsion was obtained in the same manner as in the Example 1 except that 4.08 parts by mass of [2Mabs-MA] [707-S] obtained in the Synthesis Example 2 above was mixed instead of [2A-MA] [707-S].

### Example 3

An emulsion was obtained in the same manner as in the Example 1 except that 4.08 parts by mass of [2Mabs-A] [707-S] obtained in the Synthesis Example 3 above was mixed instead of [2A-MA] [707-S] and that 0.9 part by mass of 28% ammonia water was used.

### Example 4

An emulsion was obtained in the same manner as in the Example 1 except 4.08 parts by mass of [2A-MA] [1305-S] obtained in the Synthesis Example 4 above was mixed instead of [2A-MA] [707-S].

### Example 5

An emulsion was obtained in the same manner as in the Example 1 except that 4.08 parts by mass of [2Mabs-MA] [1305-S] obtained in the Synthesis Example 5 above was mixed instead of [2A-MA] [707-S] and that 0.9 part by mass of 28% ammonia water was used.

### Example 6

An emulsion was obtained in the same manner as in the Example 1 except that 4.08 parts by mass of [2A-MA] [2320-S] obtained in the Synthesis Example 6 above was mixed instead of [2A-MA] [707-S] and that 1.0 part by mass of 28% ammonia water was used.

### Example 7

An emulsion was obtained in the same manner as in the Example 1 except that 4.08 parts by mass of [2Mabs-MA] [2320-5] obtained in the Synthesis Example 5 above was mixed instead of [2A-MA] [707-S] and that 0.9 part by mass of 28% ammonia water was used.

### Comparative Example 1

An emulsion was obtained in the same manner as in the Example 1 except that 4.08 parts by mass of 707-SF was mixed instead of [2A-MA] [707-S] and that 1.0 part by mass of 28% ammonia water was used.

### Comparative Example 2

An emulsion was obtained in the same manner as in the Example 1 except that 4.08 parts by mass of 1305-SF was mixed instead of [2A-MA] [707-S] and that 1.1 parts by mass of 28% ammonia water was used.

### Comparative Example 3

An emulsion was obtained in the same manner as in the Example 1 except that 4.08 parts by mass of 2320-SF was mixed instead of [2A-MA] [707-S] and that 1.1 parts by mass of 28% ammonia water was used.

### Comparative Example 4

An emulsion was obtained in the same manner as in the Example 1 except that 4.08 parts by mass of A-MS-60 (Antox MS-60: bis(polyoxyethylene polycyclic phenyl ether)methacrylate sulfuric acid ester salt manufactured by Nippon Nyukazai Co., Ltd.), which contains a group having a surface active ability and a reactive group in one molecule, was mixed instead of [2A-MA] [707-S] and that 1.7 parts by mass of 28% ammonia water was used.

### Example 8

Into a flask equipped with a condenser tube, a nitrogen inlet, a thermometer, a semicircular Teflon stirring blade, 100.0 parts by mass of water was introduced and heated to 80° C. Separately, 180.0 parts by mass of 2-ethylhexyl acrylate, 18.0 parts by mass of methyl methacrylate, 2.0 parts by mass of acrylic acid, 4.0 parts by mass of [2A-MA] [2320-S] synthesized in the Synthesis Example 6 above as an emulsifier, 91.9 parts by mass of water, and 10.0 parts by mass of a 10% aqueous solution of ammonium peroxodisulfate were introduced into an Erlenmeyer flask and mixed to obtain a mixture. This mixture was introduced into the above flask equipped with a condenser tube and the like by 15.3 parts by mass, and the temperature thereof was raised to 80° C. and the initial polymerization was performed for 30 minutes. Thereafter, the mixture in the Erlenmeyer flask was added into the resultant in the flask equipped with a condenser tube and the like dropwise at 80° C. over 3 hours. Further, aging of the resultant was performed at 80° C. for 1 hour, and then neutralization of the resultant was performed using 0.9 part by mass of 28% ammonia water to obtain an emulsion.

### Example 9

An emulsion was obtained in the same manner as in the Example 8 except that 4.0 parts by mass of [2Mabs-MA] [2320-S] synthesized in the Synthesis Example 7 above was mixed instead of [2A-MA] [2320-S].

### Example 10

An emulsion was obtained in the same manner as in the Example 8 except that 4.0 parts by mass of [2Mabs-MA] [707-S] synthesized in the Synthesis Example 2 above was mixed instead of [2A-MA] [2320-5] and that the amount of 28% ammonia water used was changed to 1.0 part by mass.

### Example 11

An emulsion was obtained in the same manner as in the Example 8 except that 4.0 parts by mass of [2Mabs-MA] [1305-S] synthesized in the Synthesis Example 5 above was mixed instead of [2A-MA] [2320-5] and that the amount of 28% ammonia water used was changed to 1.1 parts by mass.

### Comparative Example 5

An emulsion was obtained in the same manner as in the Example 8 except that 4.0 parts by mass of 2320-SF was mixed instead of [2A-MA] [2320-5] and that the amount of 28% ammonia water used was changed to 1.1 parts by mass.

### Comparative Example 6

An emulsion was obtained in the same manner as in the Example 8 except that 4.0 parts by mass of A-MS-60 (Antox MS-60: bis(polyoxyethylene polycyclic phenyl ether)methacrylate sulfuric acid ester salt manufactured by Nippon Nyukazai Co., Ltd.) was mixed instead of [2A-MA] [2320-S] and that the amount of 28% ammonia water used was changed to 1.3 parts by mass.

### Example 12

Into a flask equipped with a condenser tube, a nitrogen inlet, a thermometer, a semicircular Teflon stirring blade, 100.0 parts by mass of water was introduced and heated to 80° C. Separately, 80.0 parts by mass of n-butyl acrylate, 120.0 parts by mass of methyl methacrylate, 2.0 parts by mass of acrylic acid, 4.08 parts by mass of [2A-MA] [707-5] synthesized in the Synthesis Example 1 above as an emulsifier, 95.8 parts by mass of water, and 10.2 parts by mass of a 10% aqueous solution of ammonium peroxodisulfate were introduced into an Erlenmeyer flask and mixed to obtain a mixture. This mixture was introduced into the above flask equipped with a condenser tube and the like by 15.7 parts by mass, and the temperature thereof was raised to 80° C. and the initial polymerization was performed for 30 minutes. Thereafter, the mixture in the Erlenmeyer flask was added into the resultant in the flask equipped with a condenser tube and the like dropwise at 80° C. over 3 hours. Further, aging of the resultant was performed at 80° C. for 1 hour, and then neutralization of the resultant was performed using 1.5 parts by mass of 28% ammonia water to obtain an emulsion.

### Example 13

An emulsion was obtained in the same manner as in the Example 12 except that 4.08 parts by mass of [2Mabs-MA] [1305-S] synthesized in the Synthesis Example 5 above was mixed instead of [2A-MA] [707-S] and that 1.5 parts by mass of 28% ammonia water was used.

### Example 14

An emulsion was obtained in the same manner as in the Example 12 except that 4.08 parts by mass of [2Mabs-MA] [2320-5] synthesized in the Synthesis Example 7 above was mixed instead of [2A-MA] [707-S] and that 1.6 parts by mass of 28% ammonia water was used.

### Comparative Example 7

An emulsion was obtained in the same manner as in the Example 12 except that 4.08 parts by mass of A-MS-60 (Antox MS-60: bis(polyoxyethylene polycyclic phenyl ether)methacrylate sulfuric acid ester salt manufactured by Nippon Nyukazai Co., Ltd.) was mixed instead of [2A-MA] [707-S] and that 1.6 parts by mass of 28% ammonia water was used.

### Example 15

Into a flask equipped with a condenser tube, a nitrogen inlet, a thermometer, a semicircular Teflon stirring blade, 138.2 parts by mass of water was introduced and heated to 80° C. Separately, 200.0 parts by mass of styrene, 10.0 parts by mass of [2Mabs-MA] [707-S] synthesized in the Synthesis Example 2 above as an emulsifier, 138.2 parts by mass of water, and 10.0 parts by mass of a 10% aqueous solution of ammonium peroxodisulfate were introduced into an Erlenmeyer flask and mixed to obtain a mixture. Into the above flask equipped with a condenser tube and the like, 35.8 parts by mass of this mixture was introduced, and the temperature thereof was raised to 80° C. and the initial polymerization was performed for 30 minutes. Thereafter, the mixture in the Erlenmeyer flask was added into the resultant in the flask equipped with a condenser tube and the like dropwise at 80° C. over 3 hours. Further, aging of the resultant was performed at 80° C. for 2 hours, the resultant was cooled, and then neutralization thereof was performed using 0.46 part by mass of 28% ammonia water to obtain an emulsion.

### Example 16

An emulsion was obtained in the same manner as in the Example 15 except that 10.0 parts by mass of [2Mabs-MA] [1305-S] synthesized in the Synthesis Example 5 above was mixed instead of [2Mabs-MA] [707-S] and that the amount of 28% ammonia water used was changed to 0.55 part by mass.

### Example 17

An emulsion was obtained in the same manner as in the Example 15 except that 10.0 parts by mass of [2A-MA] [CMP11-S] synthesized in the Synthesis Example 8 above was mixed instead of [2Mabs-MA] [707-S] and the amount of 28% ammonia water used was changed to 0.46 part by mass.

### Example 18

An emulsion was obtained in the same manner as in the Example 15 except that 10.0 parts by mass of [2Mabs-MA] [CMP11-S] synthesized in the Synthesis Example 9 above was used instead of [2Mabs-MA] [707-S] and that the amount of 28% ammonia water used was changed to 0.60 part by mass.

### Comparative Example 8

An emulsion was obtained in the same manner as in the Example 15 except that 10.0 parts by mass of A-MS-60 (Antox MS-60: bis(polyoxyethylene polycyclic phenyl ether)methacrylate sulfuric acid ester salt manufactured by Nippon Nyukazai Co., Ltd.) was mixed instead of [2Mabs-MA] [707-S] and that the amount of 28% ammonia water used was changed to 0.56 part by mass.

### Example 19

Into a flask equipped with a condenser tube, a nitrogen inlet, a thermometer, a semicircular Teflon stirring blade, 300.0 parts by mass of methyl ethyl ketone, 105.0 parts by mass of n-butyl acrylate, 195.0 parts by mass of methyl methacrylate, and 15.0 parts by mass of [2Mabs-AA] [1305-S] synthesized in the Synthesis Example 10 above were introduced, 3.0 parts by mass of azobisisobutyronitrile was added thereto as a polymerization catalyst, and a solution polymerization was performed at 90° C. for 3 hours, thereby obtaining a resin.

### Comparative Example 9

A resin was obtained in the same manner as in the Example 19 except that 15.0 parts by mass of 1305-SF was used instead of [2Mabs-AA] [1305-S].

### Example 20

A resin was obtained in the same manner as in the Example 19 except that 15.0 parts by mass of [2Mabs-AA] [DOSS] synthesized in the Synthesis Example 11 above was used instead of [2Mabs-AA] [1305-S] and that 300 parts by mass of butyl acetate was used instead of methyl ethyl ketone, respectively.

### Comparative Example 10

A resin was obtained in the same manner as in the Example 20 except that 15.0 parts by mass of DOSS-NH₄ was used instead of [2Mabs-AA] [DOSS].

### Example 21

A resin was obtained in the same manner as in the Example 19 except that 195.0 parts by mass of styrene was used instead of methyl methacrylate.

### Comparative Example 11

A resin was obtained in the same manner as in the Example 21 except that 15.0 parts by mass of 1305-SF was used instead of [2Mabs-AA] [1305-S].

### Example 22

A resin was obtained in the same manner as in the Example 19 except that 15.0 parts by mass of [2Mabs-AA] [DOSS] synthesized in the Synthesis Example 11 above was used instead of [2Mabs-AA] [1305-S], that 300 parts by mass of butyl acetate was used instead of methyl ethyl ketone, and that 195.0 parts by mass of styrene was used instead of methyl methacrylate, respectively.

### Comparative Example 12

A resin was obtained in the same manner as in the Example 22 except that 15.0 parts by mass of DOSS-NH₄ was used instead of [2Mabs-AA] [DOSS].

(Evaluation)

[pH and Viscosity]

The pH of the emulsion thus obtained was measured using the pH METER F-51 manufactured by HORIBA, Ltd. In addition, the viscosity of the emulsion thus obtained was measured using a BL-type viscometer VISCOMETER manufactured by TOKIMEC INC.

[Emulsion Polymerization Properties]

The initial emulsion stability was evaluated as follows by visually observing the state of a liquid obtained by allowing a pre-emulsion to stand for 10 minutes.

◯: not layer separated

X: layer separated

The polymerization stability was expressed as a percentage of mass of the filtration residue after emulsion polymerization/mass of the solid content in the emulsion.

The polymerization conversion was calculated by percentage of % by mass of nonvolatile content of emulsion % by mass of theoretical nonvolatile content of emulsion (50.5%).

The average particle size of the particles in the emulsion was measured by a scattering particle size distribution analyzer LA-950 manufactured by Horiba, Ltd.

[Preparation of Specimen]

Moreover, the emulsion obtained by emulsion polymerization in Examples 1 to 7 and 12 to 14 above and Comparative Examples 1 to 4 and 7 above, or the resin solution obtained by the solution polymerization performed in Examples 19 to 22 above were coated on a glass plate at a film thickness of 75 lam and dried for 3 minutes by a dryer at 110° C., whereby a specimen having a film thickness about 15 lam was obtained. The evaluations described below were performed using this specimen.

[Water Resistance]

The water resistance was evaluated as follows by observing the state of the paint film obtained by immersing the specimen thus prepared in water for 24 hours.

State of cloudiness (water resistance)

⊙: entirely transparent

◯: partially blue cloudy

Δ: entirely blue cloudy

x: entirely cloudy Presence or absence of peeling (adhesive property)

⊙: not peeled off (not peeled off even rubbed with a finger)

◯: not peeled off (peeled off when rubbed with a finger)

Δ: partially peeled off

x: entirely peeled off

[Antistatic Properties Test (Surface Intrinsic Resistivity)]

The surface intrinsic resistivity of the specimen after production was measured at an applied voltage of 250 V using a high resistivity meter High Leicester UP and the URS probe manufactured by Mitsubishi Chemical Analytech Co., Ltd. In addition, tap water was flowed on the specimen for 30 seconds (about 1 L), subsequently the specimen was rinsed with ion exchanged water, water drop was wiped off therefrom, and then the specimen was dried for 5 minutes at 110° C. The surface intrinsic resistivity of the specimen after being subjected to this process one time was measured in the same manner as above.

Meanwhile, the term “over” in Table 1 to Table 5 means that the measured value exceeds 1×10¹³, and the indication “-” means that the measurement was not performed.

[Antifog Property Test]

The antifog property was evaluated with regard to the specimens prepared using the surfactants according to Examples 19 to 22 and Comparative Examples 9 to 12 by the presence or absence of cloudiness on the surface of the specimens as follows.

Water was introduced into a hot water layer and heated to 80° C., and then the specimen was exposed to steam for 10 seconds at a distance of 5 cm from the surface of water. The state of the specimen at that time was observed visually and evaluated according to the following two ranks.

Acceptable: not clouded at all

Unacceptable: cloudiness or water drops observed

[Leaching Rate]

The specimen was obtained by the method described in the section of preparation of specimen above. The specimen thus obtained was immersed in water at 25° C. for 1 hour or 3 hours, and then dried at 110° C. for 10 minutes. The leaching rate was calculated by the following Equation by taking the weight of the specimen before immersion as W1 and the weight of the specimen after immersion as W2 and then evaluated.

Leaching rate=(W1−W2)/W1×100 [Equation 1]

[Water Absorption Rate]

The specimen was obtained by the method described in the section of preparation of specimen above. The specimen thus obtained was immersed in water at 25° C. for 1 hour or 3 hours, and then the water drops on the specimen were rapidly wiped off with a waste cloth. The water absorption rate was calculated by the following Equation by taking the weight of the specimen before immersion as W3 and the weight of the specimen after immersion as W4 and then evaluated.

Water absorption rate=(W4−W3)/W3×100 [Equation 2]

[Foaming and Defoaming]

Into a 250 ml stoppered measuring cylinder, 10 parts by mass of the emulsion and 90 parts by mass of water were introduced and vigorously shaken for 30 seconds. The height from the surface of the liquid to the top of the foam was measured in 0.5, 1, 3, 5, and 10 minutes by taking the time after the vigorous shaking for 30 seconds as 0 second (starting point), and then evaluated.

[Chemical Stability]

To 5 parts by mass of the emulsion (Examples 1, 2, 4, 5, 6, and 7), 5 parts by mass of a 5% aqueous solution, a 10% aqueous solution, and a 15% aqueous solution of calcium chloride were respectively added and mixed, and then the presence or absence of aggregates was observed and evaluated as follows.

◯: No aggregates

Δ: aggregates observed visually and in the form of liquid

x: aggregates observed visually and in the form of gel or semi-solid

The composition of the emulsion or the reaction solution of the respective Examples and Comparative

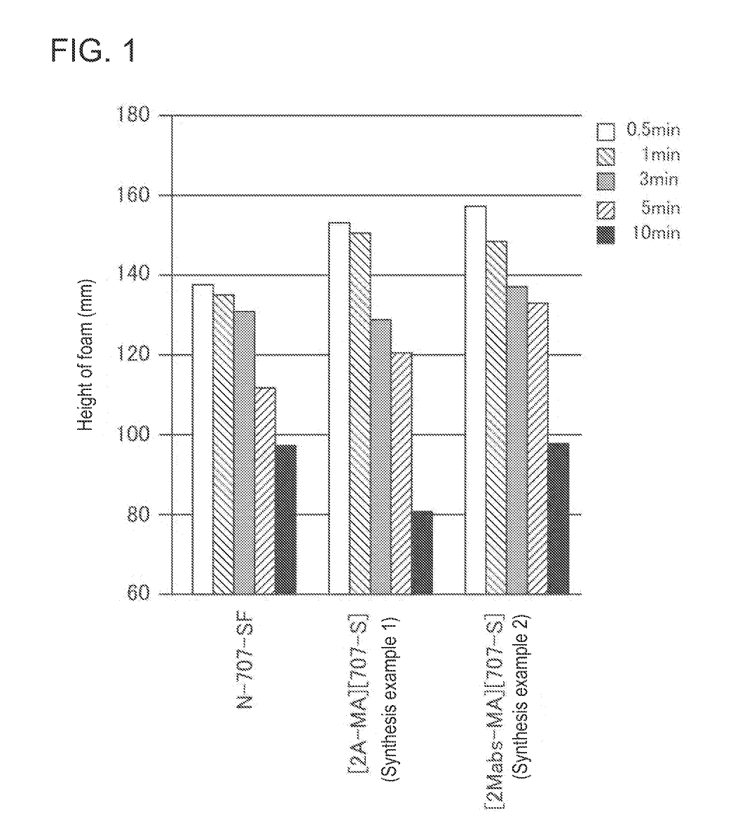

Examples and the results of the evaluations are shown in the following Table 1 to Table 5 and FIG. 1.

TABLE 1

| | Exam- | Exam- | Exam- | Comparative | Exam- | Exam- | Comparative | Exam- | Exam- | Comparative | Comparative |
|---|---|---|---|---|---|---|---|---|---|---|---|
| | ple 1 | ple 2 | ple 3 | Example 1 | ple 4 | ple 5 | Example 2 | ple 6 | ple 7 | Example 3 | Example 4 |
| Composition of monomer | | | | | | | | | | | |
| (part by mass) | | | | | | | | | | | |
| Butyl acrylate | 100 | ← | ← | ← | ← | ← | ← | ← | ← | ← | ← |
| Methyl | 70 | ← | ← | ← | ← | ← | ← | ← | ← | ← | ← |
| methacrylate | | | | | | | | | | | |
| Styrene | 30 | ← | ← | ← | ← | ← | ← | ← | ← | ← | ← |
| Acrylic acid | 4 | ← | ← | ← | ← | ← | ← | ← | ← | ← | ← |
| Emulsifier | | | | | | | | | | | |
| (part by mass) | | | | | | | | | | | |
| [2A-MA] [707-S] | 4.08 | | | | | | | | | | |
| [2Mabs-MA] [707-S] | | 4.08 | | | | | | | | | |
| [2Mabs-A] [707-S] | | | 4.08 | | | | | | | | |
| [2A-MA] [1305-S] | | | | | 4.08 | | | | | | |
| [2Mabs-MA] [1305-S] | | | | | | 4.08 | | | | | |
| [2A-MA] [2320-S] | | | | | | | | 4.08 | | | |
| [2Mabs-MA] [2320-S] | | | | | | | | | 4.08 | | |
| 707-SF | | | | 4.08 | | | | | | | |
| 1305-SF | | | | | | | 4.08 | | | | |
| 2320-SF | | | | | | | | | | 4.08 | |
| A-MS-60 | | | | | | | | | | | 4.08 |
| Polymerization initiator | | | | | | | | | | | |
| (part by mass) | | | | | | | | | | | |
| 10% aqueous solution of | 10.2 | ← | ← | ← | ← | ← | ← | ← | ← | ← | ← |
| ammonium peroxodi- | | | | | | | | | | | |
| sulfate | | | | | | | | | | | |
| Amount of monomer | 5% | 5% | 5% | 5% | 5% | 5% | 5% | 5% | 5% | 5% | 5% |
| seed (/monomer) | | | | | | | | | | | |
| Neutralizer | | | | | | | | | | | |
| (part by mass) | | | | | | | | | | | |
| 28% ammonia water | 0.8 | 0.8 | 0.9 | 1.0 | 0.8 | 0.9 | 1.1 | 1.0 | 0.9 | 1.1 | 1.7 |
| pH | 3.8 → | 3.7 → | 3.2 → | 2.2 → | 4.0 → | 4.1 → | 2.1 → | 3.4 → | 3.6 → | 2.4 → | 2.2 → |
| | 8.0 | 8.1 | 8.1 | 8.0 | 8.0 | 8.0 | 8.1 | 8.2 | 8.0 | 8.1 | 8.1 |
| Viscosity 60 rpm | | | | | | | | | | | |
| (mPa · s) | | | | | | | | | | | |
| Before | 130 | 163 | 209 | 90 | 145 | 91 | 445 | 176 | 191 | 63 | 96 |
| neutralization | | | | | | | | | | | |
| After | 169 | 216 | 288 | 101 | 173 | 107 | 245 | 271 | 195 | 140 | 107 |
| neutralization | | | | | | | | | | | |
| Initial emulsion | ◯ | ◯ | ◯ | ◯ | ◯ | ◯ | ◯ | ◯ | ◯ | ◯ | ◯ |
| stability | | | | | | | | | | | |
| Polymerization | 0.01 | 0.05 | 0.03 | 0.01 | 0.05 | 0 | 0.05 | 0.05 | 0.05 | 0.04 | 0.414 |
| stability (%) | | | | | | | | | | | |
| Polymerization | 99.8 | 98.2 | 99.5 | 100 | 99.6 | 99.5 | 99.8 | 99.2 | 99.2 | 99.7 | 99.7 |
| conversion (%) | | | | | | | | | | | |
| Average particle | 0.08 | 0.08 | 0.07 | 0.13 | 0.08 | 0.1 | 0.08 | 0.08 | 0.07 | 0.1 | 0.12 |
| size (μm) | | | | | | | | | | | |
| Water resistance | ⊙ | ⊙ | ⊙ | X | ⊙ | ◯ | Δ | ⊙ | ⊙ | X | ◯ |
| Chemical stability | | | | | | | | | | | |
| 5% | ◯ | ◯ | — | ◯ | ◯ | ◯ | ◯ | ◯ | ◯ | ◯ | ◯ |
| 10% | X | Δ | — | Δ | X | X | X | Δ | ◯ | ◯ | X |
| Water absorption rate | | | | | | | | | | | |
| (% by mass) | | | | | | | | | | | |
| 1 hour | 4.78 | — | — | 8.36 | 5.55 | — | — | 4.43 | — | — | — |
| 3 hours | 8.46 | — | — | 9.89 | 6.86 | — | — | 9.22 | — | — | — |
| Leaching rate | | | | | | | | | | | |
| (% by mass) | | | | | | | | | | | |
| 1 hour | 0.11 | — | — | 0.11 | 0.20 | — | 1.12 | 0.15 | — | 1.19 | — |
| 3 hours | 0.34 | — | — | 0.61 | 0.28 | — | 1.25 | 0.21 | — | 1.22 | — |
| Adhesive property | ◯ | ◯ | ◯ | X | ◯ | ◯ | X | ◯ | ◯ | ◯ | ◯ |
| Surface resistivity | over | over | over | over | over | over | 2 × 10¹⁰ | over | over | over | over |
| (Ω/•) | | | | | | | | | | | |
| Surface resistivity | — | — | — | — | — | — | over | — | — | — | — |
| after washing with | | | | | | | | | | | |
| water (Ω/•) | | | | | | | | | | | |

TABLE 2

| | | | | | Comparative | Comparative |
|---|---|---|---|---|---|---|
| | Example 8 | Example 9 | Example 10 | Example 11 | Example 5 | Example 6 |
| Composition of monomer | | | | | | |
| (part by mass) | | | | | | |
| 2-Ethylhexyl acrylate | 180 | ← | ← | ← | ← | ← |
| Methyl | 18 | ← | ← | ← | ← | ← |
| methacrylate | | | | | | |
| Acrylic acid | 2 | ← | ← | ← | ← | ← |
| Emulsifier | | | | | | |
| (part by mass) | | | | | | |
| [2A-MA] [2320-S] | 4 | | | | | |
| [2Mabs-MA] [2320-S] | | 4 | | | | |
| [2Mabs-MA] [707-S] | | | 4 | | | |
| [2Mabs-MA] [1305-S] | | | | 4 | | |
| 2320-SF | | | | | 4 | |
| A-MS-60 | | | | | | 4 |
| Polymerization initiator | | | | | | |
| (part by mass) | | | | | | |
| 10% aqueous solution of | 10.2 | ← | ← | ← | ← | ← |
| ammonium peroxodisulfate | | | | | | |
| Amount of monomer seed | 5% | 5% | 5% | 5% | 5% | 5% |
| (/monomer) | | | | | | |
| Neutralizer | | | | | | |
| (part by mass) | | | | | | |
| 28% ammonia water | 0.9 | 0.9 | 1.0 | 1.1 | 1.1 | 1.3 |
| pH | 2.47 → | 2.59 → | 3.54 → | 3.15 → | 1.99 → | 1.80 → |
| | 6.58 | 6.59 | 6.51 | 6.55 | 6.59 | 6.58 |
| Viscosity 60 rpm | | | | | | |
| (mPa · s) | | | | | | |
| Before | 100 | 235 | 252 | 178 | 350 | 147 |
| neutralization | | | | | | |
| After | 107 | 285 | 278 | 180 | 320 | 282 |
| neutralization | | | | | | |
| Initial emulsion | ◯ | ◯ | ◯ | ◯ | ◯ | ◯ |
| stability | | | | | | |
| Polymerization | 0.253 | 0.085 | 0 | 0 | 0.015 | 0.788 |
| stability (%) | | | | | | |
| Polymerization | 100 | 100 | 99.7 | 99 | 100 | 99.9 |
| conversion (%) | | | | | | |
| Average particle | 0.14 | 0.09 | 0.09 | 0.10 | 0.11 | 0.10 |
| size (μm) | | | | | | |

TABLE 3

| | | | | Comparative |
|---|---|---|---|---|
| | Example 12 | Example 13 | Example 14 | Example 7 |
| Composition of monomer | | | | |
| (part by mass) | | | | |
| Methyl methacrylate | 120 | ← | ← | ← |
| Butyl acrylate | 80 | ← | ← | ← |
| Acrylic acid | 2 | ← | ← | ← |
| Emulsifier | | | | |
| (part by mass) | | | | |
| [2Mabs-MA] [707-S] | 4.08 | | | |
| [2Mabs-MA] [1305-S] | | 4.08 | | |
| [2Mabs-MA] [2320-S] | | | 4.08 | |
| A-MS-60 | | | | 4.08 |
| Polymerization initiator | | | | |
| (part by mass) | | | | |
| 10% aqueous solution of | 10.2 | ← | ← | ← |
| ammonium peroxodisulfate | | | | |
| Amount of monomer seed | 5% | 5% | 5% | 5% |
| (/monomer) | | | | |
| Neutralizer | | | | |
| (part by mass) | | | | |
| 28% ammonia water | 1.5 | 1.5 | 1.6 | 1.6 |
| pH | 3.7 → | 3.8 → | 2.6 → | 1.8 → |
| | 8.1 | 8.1 | 8.0 | 8.0 |
| Viscosity 60 rpm | | | | |
| (mPa · s) | | | | |
| Before neutralization | 122 | 95 | 96 | 96 |
| After neutralization | 164 | 106 | 106 | 106 |
| Initial emulsion | ◯ | ◯ | ◯ | ◯ |
| stability | | | | |
| Polymerization | 0.02 | 0.000 | 0.21 | 5.47 |
| stability (%) | | | | |
| Polymerization | 98.8 | 100 | 99.6 | 98.3 |
| conversion (%) | | | | |
| Average particle size | 0.08 | 0.09 | 0.08 | 0.14 |
| (μm) | | | | |
| Water resistance | ◯ | ◯ | ◯ | X |
| Surface resistivity | — | 5 × 10¹⁰ | — | — |
| (Ω/•) | | | | |

TABLE 4

| | | | | | Comparative |
|---|---|---|---|---|---|
| | Example 15 | Example 16 | Example 17 | Example 18 | Example 8 |
| Composition of monomer | | | | | |
| (part by mass) | | | | | |
| Styrene | 200 | ← | ← | ← | ← |
| Emulsifier | | | | | |
| (part by mass) | | | | | |
| [2Mabs-MA] [707-S] | 10.0 | | | | |
| [2Mabs-MA] [1305-S] | | 10.0 | | | |
| [2A-MA] [CMP11-S] | | | 10.0 | | |
| [2Mabs-MA] [CMP11-S] | | | | 10.0 | |
| A-MS-60 | | | | | 10.0 |
| Polymerization initiator | | | | | |
| (part by mass) | | | | | |
| 10% aqueous solution of | 10.0 | ← | ← | ← | ← |
| ammonium peroxodisulfate | | | | | |
| Amount of monomer seed | 5% | ← | ← | ← | ← |
| (/monomer) | | | | | |
| Neutralizer | | | | | |
| (part by mass) | | | | | |
| 28% ammonia water | 0.46 | 0.55 | 0.46 | 0.60 | 0.56 |
| pH | 5.7 → | 5.7 → | 2.0 → | 2.0 → | 1.8 → |
| | 8.2 | 8.1 | 8.0 | 8.0 | 8.0 |
| Viscosity 60 rpm | | | | | |
| (mPa · s) | | | | | |
| Before | 26 | 24 | 22 | 22 | 27 |
| neutralization | | | | | |
| After | 26 | 24 | 22 | 22 | 27 |
| neutralization | | | | | |
| Initial emulsion | ◯ | ◯ | ◯ | ◯ | ◯ |
| stability | | | | | |
| Polymerization | 0.18 | 0.045 | 0.014 | 0.008 | 0.365 |
| stability (%) | | | | | |
| Polymerization | 99.1 | 99.0 | 99.3 | 99.7 | 99.6 |
| conversion (%) | | | | | |
| Average particle | 0.07 | 0.07 | 0.07 | 0.07 | 0.07 |
| size (μm) | | | | | |

TABLE 5

| | | Comparative | | Comparative | | Comparative | | Comparative |
|---|---|---|---|---|---|---|---|---|
| | Example 19 | Example 9 | Example 20 | Example 10 | Example 21 | Example 11 | Example 22 | Example 12 |
| Composition of monomer | | | | | | | | |
| (part by mass) | | | | | | | | |
| Methyl methacrylate | 195 | ← | ← | ← | | | | |
| Styrene | | | | | 195 | ← | ← | ← |
| Butyl acrylate | 105 | ← | ← | ← | ← | ← | ← | ← |
| Solvent | | | | | | | | |
| Methyl ethyl ketone | 300 | ← | | | 300 | ← | | |
| Butyl acetate | | | 300 | ← | | | 300 | ← |
| Dispersant | | | | | | | | |
| (part by mass) | | | | | | | | |
| [2Mabs-AA] [1305-S] | 15 | | | | 15 | | | |
| [2Mabs-AA] [DOSS] | | | 15 | | | | 15 | |
| DOSS-NH₄ | | | | 15 | | | | 15 |
| 1305-SF | | 15 | | | | 15 | | |
| Polymerization initiator | | | | | | | | |
| (part by mass) | | | | | | | | |
| Azobisisobutyronitrile | 3 | ← | ← | ← | ← | ← | ← | ← |
| Surface resistivity | | | | | | | | |
| After washing with | 1 × 10¹⁰ | 5 × 10¹¹ | 7 × 10¹⁰ | 5 × 10¹⁰ | 2 × 10⁹ | 5 × 10¹¹ | 1 × 10¹¹ | 5 × 10¹⁰ |
| water 0 time | | | | | | | | |
| After washing with | 4 × 10¹⁰ | over | 7 × 10¹¹ | over | 5 × 10⁹ | over | 3 × 10¹¹ | over |
| water 1 time | | | | | | | | |
| Antifog property | | | | | | | | |
| After washing with | Acceptable | Unacceptable | Acceptable | Acceptable | — | Unacceptable | Acceptable | Acceptable |
| water 0 time | | | | | | | | |
| After washing with | Acceptable | Unacceptable | Acceptable | Unacceptable | — | Unacceptable | — | Unacceptable |
| water 1 time | | | | | | | | |

As can be seen from Tables 1 to 4, high initial polymerization stability, polymerization conversion, and polymerization stability are obtained in the emulsion polymerization performed using the ionically bonded salt having a reactive group of the invention in all of the cases in which the monomer is composed of only an acrylate-based monomer, only styrene, and a mixed composition thereof. In addition, the average particle size of the resin obtained in these Examples are suppressed to be smaller compared to that of the A-MS-60 which is used in Comparative Examples and a surfactant containing a group having a surface active ability and a reactive group in one molecule (Comparative Example 4 and Comparative Examples 6 to 8) or the anionic surfactant (Comparative Examples 1 to 5). In particular, the polymerization stability in Examples is significantly improved compared to the case of using the A-MS-60.

In addition, as can be seen from Table 1 and Table 3 above, the coating film obtained from the thermoplastic resin composition containing the ionically bonded salt having a reactive group of the invention are excellent in water resistance and adhesive property compared to that obtained from the thermoplastic resin composition containing the anionic surfactant used in Comparative Examples 1 to 3 or the surfactant containing a group having a surface active ability and a reactive group in one molecule used in Comparative Examples 4 and 7, and in particular, the water resistance is significantly improved. The result with regard to the water resistance is also supported by the fact that the water absorption rate of the coating film obtained from the thermoplastic resin composition containing the ionically bonded salt having a reactive group of the invention is lower compared to that of the coating film obtained from the thermoplastic resin composition obtained from an anionic surfactant shown in Table 1. In addition, as can be seen from Table 1, the leaching rate of the coating film obtained from the thermoplastic resin composition containing the ionically bonded salt having a reactive group of the invention is suppressed to be lower compared to that of Comparative Example 1. This fact leads to the reduction of the environmental impact since the amount of surfactant remaining in the waste liquid after polymerization can be reduced.

In addition, as can be seen from Table 3, [2Mabs-MA] [1305-S] exhibits a sufficiently low surface resistivity as an antistatic agent, and thus the ionically bonded salt according to the invention is also applicable to the application as an antistatic agent. Moreover, as can be seen from Table 5, a surface resistivity value low enough to be used as an antistatic agent were obtained even in the coating film using the resin obtained by a solution polymerization. In particular, [2Mabs-AA] [1305-S] exhibits excellent antistatic performance compared to 1305-SF of an anionic surfactant which does not have a reactive cation, and exhibits excellent water resistance as well.

As described above, the coating film containing the ionically bonded salt according to the invention is excellent in antistatic performance as well as water resistance and weather resistance which are important properties for a coating film.

In addition, as can be seen from the comparison on the antifog property of Example 19 with Comparative Example 9 and Example 20 with Comparative Example 10 shown in Table 5, the ionically bonded liquid according to the invention exhibits superior antifog property to the corresponding anionic surfactant, and is excellent in water resistance as well.

Moreover, as can be seen from FIG. 1, the defoaming property excellently exhibited by the anionic surfactant such as N-707-SF is maintained by the ionically bonded salt of the invention having a cation having a reactive group as well.

Moreover, the present application is based upon the prior Japanese Patent Application No. 2012-44318, filed on Feb. 29, 2012, Japanese Patent Application No. 2012-119508, filed on May 25, 2012, and Japanese Patent application No. 2012-237048, filed on Oct. 26, 2012, the entire contents of which are incorporated herein by reference.

1-3. (canceled)

4. A treatment agent, which is an antistatic agent and comprises an ionically bonded salt having a reactive group and is represented by the following Chemical Formula (1) or the following Chemical Formula (2): 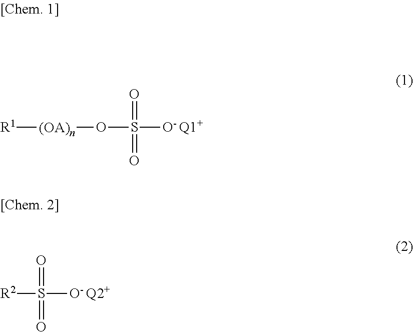
in Chemical Formulas (1) and (2) above, R¹ and R² each independently represent a substituted or unsubstituted linear, branched, or cyclic alkyl group having from 1 to 30 carbon atoms, a substituted or unsubstituted aryl group having from 6 to 30 carbon atoms, or a substituted or unsubstituted arylalkyl group having from 7 to 31 carbon atoms, A is a linear or branched alkylene group having from 2 to 4 carbon atoms, n represents an integer of 0 to 50, and Q1 and Q2 each independently represent at least one kind selected from the group consisting of an ammonium ion having an ethylenically unsaturated bond, an imidazolium ion having an ethylenically unsaturated bond, a pyridinium ion having an ethylenically unsaturated bond, a pyrrolidinium on having an ethylenically unsaturated bond, a pyrrolinium ion having an ethylenically unsaturated bond, a piperidinium ion having an ethylenically unsaturated bond, a pyrazinium ion having an ethylenically unsaturated bond, a pyrimidinium ion having an ethylenically unsaturated bond, a triazolium ion having an ethylenically unsaturated bond, a triazinium ion having an ethylenically unsaturated bond, a quinolinium ion having an ethylenically unsaturated bond, an isoquinolinium ion having an ethylenically unsaturated bond, an indolinium ion having an ethylenically unsaturated bond, a quinoxalinium ion having an ethylenically unsaturated bond, a piperazinium ion having an ethylenically unsaturated bond, an oxazolinium ion having an ethylenically unsaturated bond, a thiazolinium ion having an ethylenically unsaturated bond, and a morpholinium ion having an ethylenically unsaturated bond.

5. A treatment agent, which is an antifogging agent and comprises an ionically bonded salt having a reactive group and is represented by the following Chemical Formula (1) or the following Chemical Formula (2): 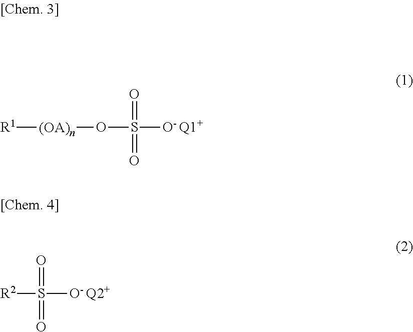
in Chemical Formulas (1) and (2) above, R¹ and R², A, n, and Q1 and Q2 have the same definitions as above.

6. A treatment agent 3, which is a dispersant or an emulsifier and comprises an ionically bonded salt having a reactive group and is represented by the following Chemical Formula (1) or the following Chemical Formula (2): 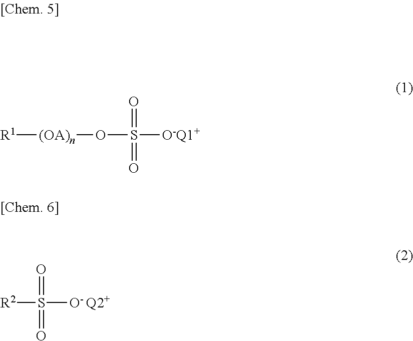
in Chemical Formulas (d (2) above, R¹ and R², n, and Q1 and Q2 have the same definitions as above.

7. A treatment agent, which is a weathering agent and comprises an ionically bonded salt having a reactive group and is represented by the following Chemical Formula (1) or the following Chemical Formula (2): 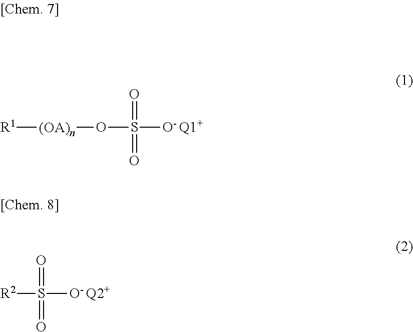
in Chemical Formulas (1) and (2) above, R¹ and R², n, and Q1 and Q2 have the same definitions as above.

8. A thermoplastic resin composition comprising the ionically bonded salt having a reactive group and is represented by the following Chemical Formula (1) or the following Chemical Formula (2) and a thermoplastic resin, wherein the content of the ionically bonded salt having a reactive group is from 0.1 to 20 parts by mass with respect to 100 parts by mass of the thermoplastic resin: 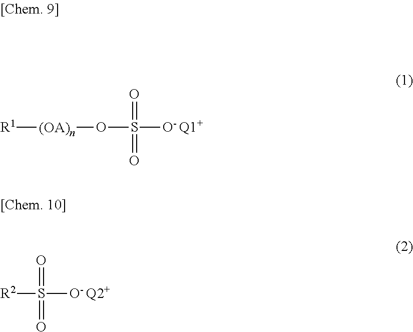
in Chemical Formulas (1) and (2) above, R¹ and R², A, n, and Q1 and Q2 have the same definitions as above.

9. (canceled)

10. The thermoplastic resin composition according to claim 8, wherein the thermoplastic resin is at least one kind selected from the group consisting of a (meth)acrylic resin, a styrene resin, an olefin resin, and a polyester resin

11. The thermoplastic resin composition according to claim 8 or 10, which is in the form of an emulsion.

12. The treatment agent according to any one of claims 4 to 7, wherein Q1 and Q2 in Chemical Formulas (1) and (2) above each independently represent at least one kind selected from the group consisting of an ammonium ion having an ethylenically unsaturated bond and a piperidinium ion having an ethylenically unsaturated bond.
